(19) United States
(12) Patent Application Publication
(10) Pub. No.: US 2010/0160385 A1
(43) Pub. Date: Jun. 24, 2010

(54) FUNGICIDAL TRIPHENYL-SUBSTITUTED PYRIDONES

(75) Inventor: Bruce Lawrence Finkelstein, Newark, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E.I. DU PONT DE NEMOURS AND COMPANY, Wilmington, DE (US)

(21) Appl. No.: 12/621,903

(22) Filed: Nov. 19, 2009

Related U.S. Application Data
(60) Provisional application No. 61/139,027, filed on Dec. 19, 2008.

(51) Int. Cl. A01N 43/40; C07D 213/63; A01P 3/00

(52) U.S. Cl. 514/345; 546/290

(57) ABSTRACT

Disclosed are compounds of Formula 1, including all geometric and stereoisomers, N-oxides, and salts thereof,

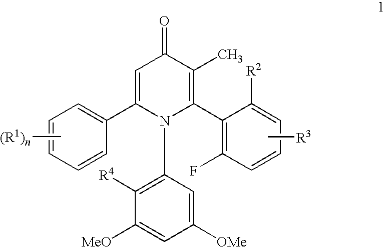

wherein
R¹, R², R³, R⁴ and n are as defined in the disclosure.
   Also disclosed are compositions containing the compounds of Formula 1 and methods for controlling plant disease caused by a fungal pathogen comprising applying an effective amount of a compound or a composition of the invention.

### FIELD OF THE INVENTION

This invention relates to certain substituted pyridones, their N-oxides, salts and compositions, and methods of their use as fungicides.

### BACKGROUND OF THE INVENTION

The control of plant diseases caused by fungal plant pathogens is extremely important in achieving high crop efficiency. Plant disease damage to ornamental, vegetable, field, cereal, and fruit crops can cause significant reduction in productivity and thereby result in increased costs to the consumer. Many products are commercially available for these purposes, but the need continues for new compounds which are more effective, less costly, less toxic, environmentally safer or have different sites of action.

U.S. Pat. No. 4,757,081 discloses certain 1,2,6-triphenyl-4(1H)-pyridinone derivatives of Formula i

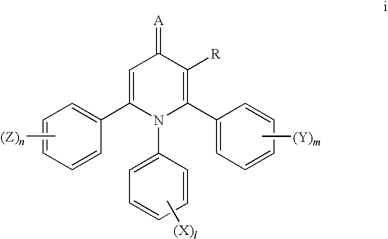

and their use as fungicides.

European Patent 304057 discloses pyridinone derivatives of Formula ii

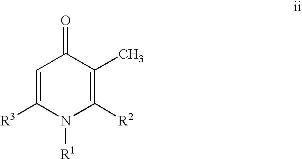

and their use as fungicides.

### SUMMARY OF THE INVENTION

This invention is directed to compounds of Formula 1 (including all geometric and stereoisomers), N-oxides, and salts thereof, agricultural compositions containing them and their use as fungicides:

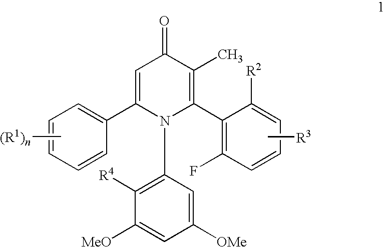

wherein
each R¹ is independently halogen, cyano, hydroxy, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₃-C₆ cycloalkenyl, C₂-C₆ alkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₃-C₆ dialkylaminoalkyl, C₂-C₆ alkylcarbonyl, C₂-C₆ haloalkylcarbonyl, C₂-C₆ alkoxycarbonyl, C₂-C₆ alkylaminocarbonyl, C₃-C₈ dialkylaminocarbonyl, C₂-C₆ cyanoalkyl, C₁-C₆ hydroxyalkyl, C₁-C₆ alkoxy, C₁-C₆ haloalkoxy, C₃-C₆ cycloalkoxy, C₃-C₆ halocycloalkoxy, C₂-C₆ alkoxyalkoxy, C₃-C₆ alkoxycarbonylalkyl, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₁-C₆ alkylamino, C₂-C₆ dialkylamino, C₂-C₆ haloalkylamino, C₂-C₆ halodialkylamino, C₂-C₆ alkylcarbonylamino, C₂-C₆ haloalkylcarbonylamino, C₁-C₆ alkylsulfonylamino or C₁-C₆ haloalkylsulfonylamino;
R² is F or Cl;
R³ is halogen or methoxy;
R⁴ is H or halogen; and
n is an integer selected from 0, 1, 2, 3, 4 and 5.

More particularly, this invention pertains to a compound of Formula 1 (including all geometric and stereoisomers), an N-oxide, or a salt thereof.

This invention also relates to a fungicidal composition comprising a compound of Formula 1 and at least one additional component selected from the group consisting of surfactants, solid diluents and liquid diluents.

This invention also relates to a fungicidal composition comprising a mixture of a compound of Formula 1 and at least one other fungicide (e.g., at least one other fungicide having a different site of action).

This invention further relates to a method for controlling plant diseases caused by fungal plant pathogens comprising applying to the plant or portion thereof, or to the plant seed, a fungicidally effective amount of a compound of the invention (e.g., as a composition described herein).

### DETAILS OF THE INVENTION

As used herein, the terms “comprises,” “comprising,” “includes,” “including,” “has,” “having”, “contains” or “containing” or any other variation thereof, are intended to cover a non-exclusive inclusion. For example, a composition, process, method, article, or apparatus that comprises a list of elements is not necessarily limited to only those elements but may include other elements not expressly listed or inherent to such composition, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in the present disclosure and claims, “plant” includes members of Kingdom Plantae, particularly seed plants (Spermatopsida), at all life stages, including young plants (e.g., germinating seeds developing into seedlings) and mature, reproductive stages (e.g., plants producing flowers and seeds). Portions of plants include geotropic members typically growing beneath the surface of the growing medium (e.g., soil), such as roots, tubers, bulbs and corms, and also members growing above the growing medium, such as foliage (including stems and leaves), flowers, fruits and seeds.

As referred to herein, the term “seedling”, used either alone or in a combination of words means a young plant developing from the embryo of a seed.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl.

“Alkoxy” includes, for example, methoxy, ethoxy, n-propyloxy, isopropyloxy and the different butoxy, pentoxy and hexyloxy isomers. “Alkylthio” includes branched or straight-chain alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(═O), CH₃CH₂S(═O), CH₃CH₂CH₂S(═O), (CH₃)₂CHS(═O) and the different butylsulfinyl, pentylsulfinyl and hexylsulfinyl isomers. Examples of “alkylsulfonyl” include CH₃S(═O)₂, CH₃CH₂S(═O)₂, CH₃CH₂CH₂S(═O)₂, (CH₃)₂CHS(═O)₂, and the different butylsulfonyl, pentylsulfonyl and hexylsulfonyl isomers. “Alkylamino” includes an NH radical substituted with straight-chain or branched alkyl. Examples of “alkylamino” include CH₃CH₂NH, CH₃CH₂CH₂NH, and (CH₃)₂CHCH₂NH. Examples of “dialkylamino” include (CH₃)₂N, (CH₃CH₂CH₂)₂N and CH₃CH₂(CH₃)N.

“Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Alkoxyalkoxy” denotes alkoxy substitution on alkoxy.

“Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂CH₂SCH₂ and CH₃CH₂SCH₂CH₂; “alkylsulfinylalkyl” and “alkylsulfonylalkyl” include the corresponding sulfoxides and sulfones, respectively. “Alkylaminoalkyl” denotes alkylamino substitution on alkyl. Examples of “alkylaminoalkyl” include CH₃NHCH₂, CH₃NHCH₂CH₂, CH₃CH₂NHCH₂, CH₃CH₂CH₂CH₂NHCH₂ and CH₃CH₂NHCH₂CH₂. Examples of “dialkylaminoalkyl” include ((CH₃)₂CH)₂NCH₂, (CH₃CH₂CH₂)₂NCH₂ and CH₃CH₂(CH₃)NCH₂CH₂.

“Cyanoalkyl” denotes an alkyl group substituted with one cyano group. Examples of “cyanoalkyl” include NCCH₂, NCCH₂CH₂ and CH₃CH(CN)CH₂.

“Cycloalkyl” includes, for example, cyclopropyl, cyclobutyl, cyclopentyl and cyclohexyl. The term “alkylcycloalkyl” denotes alkyl substitution on a cycloalkyl moiety and includes, for example, ethylcyclopropyl, i-propylcyclobutyl, 3-methylcyclopentyl and 4-methylcyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. The term “cycloalkoxy” denotes cycloalkyl linked through an oxygen atom such as cyclopentyloxy and cyclohexyloxy. “Alkylcycloalkylalkyl” denotes an alkyl group substituted with alkylcycloalkyl. Examples of “alkylcycloalkylalkyl” include 1-, 2-, 3- or 4-methyl or -ethyl cyclohexylmethyl. “Cycloalkenyl” includes groups such as cyclopentenyl and cyclohexenyl as well as groups with more than one double bond such as 1,3- and 1,4-cyclohexadienyl.

“Alkylcarbonyl” denotes a straight-chain or branched alkyl moieties bonded to a C(═O) moiety. Examples of “alkylcarbonyl” include CH₃C(═O), CH₃CH₂CH₂C(═O) and (CH₃)₂CHC(═O). Examples of “alkoxycarbonyl” include CH₃OC(═O), CH₃CH₂OC(═O), CH₃CH₂CH₂C(═O), (CH₃)₂CHOC(═O) and the different butoxy- or pentoxycarbonyl isomers. Examples of “alkylaminocarbonyl” include CH₃NHC(═O), CH₃CH₂NHC(═O), CH₃CH₂CH₂NHC(═O), (CH₃)₂CHNHC(═O) and the different butylamino- or pentylaminocarbonyl isomers. Examples of “dialkylaminocarbonyl” include (CH₃)₂NC(═O), (CH₃CH₂)₂NC(═O), CH₃CH₂(CH₃)NC(═O), (CH₃)₂CH(CH₃)NC(═O) and CH₃CH₂CH₂(CH₃)NC(═O). “Alkoxycarbonylalkyl” denotes alkoxycarbonyl substitution on straight-chain or branched alkyl. Examples of “alkoxycarbonylalkyl” include CH₃C(═O)CH₂CH(CH₃), CH₃CH₂C(═O)CH₂CH₂ and (CH₃)₂CHOC(═O)CH₂.

The term “alkylcarbonylamino” denotes alkyl bonded to a C(═O)NH moiety. Examples of “alkylcarbonylamino” include CH₃CH₂C(═O)NH and CH₃CH₂CH₂C(═O)NH. “Alkylsulfonylamino” denotes an NH radical substituted with alkylsulfonyl. Examples of “alkylsulfonylamino” include CH₃CH₂S(═O)₂NH and (CH₃)₂CHS(═O)₂NH.

“Trialkylsilyl” includes 3 branched and/or straight-chain alkyl radicals attached to and linked through a silicon atom, such as trimethylsilyl, triethylsilyl and tert-butyldimethylsilyl.

“Hydroxyalkyl” denotes an alkyl group substituted with one hydroxy group. Examples of “hydroxyalkyl” include HOCH₂CH₂, CH₃CH₂(OH)CH and HOCH₂CH₂CH₂CH₂.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C, ClCH₂, CF₃CH₂ and CF₃CCl₂. The terms “halocycloalkyl”, “haloalkoxy”, “haloalkylthio”, “haloalkylamino”, “haloalkylsulfinyl”, “haloalkylsulfonyl”, “haloalkenyl”, “haloalkynyl”, and the like, are defined analogously to the term “haloalkyl”. Examples of “halocycloalkyl” include 2-chlorocyclopropyl, 2-fluorocyclobutyl, 3-bromocyclopentyl and 4-chorocyclohexyl. Examples of “haloalkoxy” include CF₃O, CCl₃CH₂O, HCF₂CH₂CH₂O and CF₃CH₂O. Examples of “haloalkylthio” include CCl₃S, CF₃S, CCl₃CH₂S and ClCH₂CH₂CH₂S. Examples of “haloalkylamino” include CF₃(CH₃)CHNH, (CF₃)₂CHNH and CH₂ClCH₂NH. Examples of “haloalkylsulfinyl” include CF₃S(═O), CCl₃S(═O), CF₃CH₂S(═O) and CF₃CF₂S(═O). Examples of “haloalkylsulfonyl” include CF₃S(═O)₂, CCl₃S(═O)₂, CF₃CH₂S(═O)₂ and CF₃CF₂S(═O)₂. Examples of “haloalkenyl” include (Cl)₂C═CHCH₂ and CF₃CH₂CH═CHCH₂. Examples of “haloalkynyl” include HC≡CCHCl, CF₃C≡C, CCl₃C≡C and FCH₂C≡CCH₂. The term “halodialkyl”, either alone or in compound words such as “halodialkylamino”, means at least one of the two alkyl groups is substituted with at least one halogen atom, and independently each halogenated alkyl group may be partially or fully substituted with halogen atoms which may be the same or different. Examples of “halodialkylamino” include (BrCH₂CH₂)₂N and BrCH₂CH₂(ClCH₂CH₂)N.

The total number of carbon atoms in a substituent group is indicated by the “Cᵢ-Cⱼ” prefix where i and j are numbers from 1 to 9. For example, C₁-C₄ alkylsulfonyl designates methylsulfonyl through butylsulfonyl; C₂ alkoxyalkyl designates CH₃OCH₂; C₃ alkoxyalkyl designates, for example, CH₃CH(OCH₃), CH₃OCH₂CH₂ or CH₃CH₂OCH—; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂.

When a compound is substituted with a substituent bearing a subscript that indicates the number of said substituents can vary, then when the number of said substituents is greater than 1, said substituents are independently selected from the group of defined substituents (e.g., (R¹)ₙ wherein n is 1, 2, 3, 4 or 5).

Compounds of this invention can exist as one or more stereoisomers. The various stereoisomers include enantiomers, diastereomers, atropisomers and geometric isomers. Of note are atropisomers, which are conformational isomers that occur when rotation about a single bond in a molecule is restricted as a result of steric interaction with other parts of the molecule and the substituents at both ends of the single bond are unsymmetrical. In the present invention, atropisomerism occurs at a single bond in Formula 1 when the rotational barrier is high enough (about ΔG>25 kcal mol⁻¹) that separation of isomers at ambient temperature becomes possible. One skilled in the art will appreciate that one atropisomer may be more active and/or may exhibit beneficial effects when enriched relative to the other atropisomer or when separated from the other atropisomer. Additionally, the skilled artisan knows how to separate, enrich, and/or to selectively prepare said atropisomers. A detailed description of atropisomers can be found in March, Advanced Organic Chemistry, 101-102, 4^{th} Ed. 1992 and Gawronski et al, Chirality 2002, 14, 689-702. This invention includes compounds or compositions that are enriched in an atropisomer of Formula 1 compared to other atropisomers of the compounds. Also included are the essentially pure atropisomers of compounds of Formula 1.

Compounds of this invention can exist as one or more conformational isomers due to restricted rotation about an amide bond (e.g., wherein R¹ is alkylaminocarbonyl or dialkylaminocarbonyl). This invention comprises mixtures of conformational isomers. In addition, this invention includes compounds that are enriched in one conformer relative to others.

One skilled in the art will recognize that tertiary amines can form N-oxides. Synthetic methods for the preparation of N-oxides of tertiary amines are very well known by one skilled in the art including the oxidation of tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of plant diseases caused by fungal plant pathogens (i.e. are agriculturally suitable). The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety such as a phenol, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from Formula 1, N-oxides and agriculturally suitable salts thereof.

Compounds selected from Formula 1, geometric and stereoisomers, N-oxides, and salts thereof, typically exist in more than one form, and Formula 1 thus includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures.

Embodiments of the present invention as described in the Summary of the Invention include those described below. In the following Embodiments, Formula 1 includes geometric and stereoisomers, N-oxides, and salts thereof, and reference to “a compound of Formula 1” includes the definitions of substituents specified in the Summary of the Invention unless further defined in the Embodiments.
Embodiment 1. A compound of Formula 1 wherein each R¹ is independently halogen, cyano, nitro, C₁-C₆ alkyl, C₂-C₆ alkenyl, C₂-C₆ alkynyl, C₁-C₆ haloalkyl, C₂-C₆ alkoxyalkyl, C₂-C₆ alkylcarbonyl, C₂-C₆ alkoxycarbonyl, C₂-C₆ alkylaminocarbonyl, C₃-C₈ dialkylaminocarbonyl, C₂-C₆ cyanoalkyl, C₁-C₆ hydroxyalkyl, C₁-C₆ alkoxy, C₁-C₆ alkylthio, C₁-C₆ alkylsulfinyl, C₁-C₆ alkylsulfonyl, C₁-C₆ alkylamino, C₂-C₆ dialkylamino or C₂-C₆ alkylcarbonylamino.
Embodiment 2. A compound of Embodiment 1 wherein each R¹ is independently halogen, C₁-C₆ alkyl, C₂-C₆ alkenyl, C₁-C₆ haloalkyl, C₂-C₆ alkylcarbonyl, C₂-C₆ alkoxycarbonyl or C₁-C₆ alkoxy.
Embodiment 3. A compound of Embodiment 2 wherein each R¹ is independently halogen, C₁-C₆ alkyl or C₁-C₆ alkoxy.
Embodiment 4. A compound of Embodiment 3 wherein each R¹ is independently halogen.
Embodiment 4a. A compound of Embodiment 4 wherein each R¹ is independently F or Cl.
Embodiment 5. A compound of Formula 1 or any one of Embodiments 1 through 4a wherein R² is F.
Embodiment 6. A compound of Formula 1 or any one of Embodiments 1 through 5 wherein R³ is Cl, F, Br or methoxy.
Embodiment 7. A compound of Embodiment 6 wherein R³ is F or methoxy.
Embodiment 8. A compound of Embodiment 7 wherein R³ is methoxy.
Embodiment 9. A compound of Embodiment 7 wherein R³ is F.
Embodiment 10. A compound of Formula 1 or any one of Embodiments 1 through 9 wherein the R³ substituent is attached at the para position.
Embodiment 11. A compound of Formula 1 or any one of Embodiments 1 through 10 wherein R⁴ is H, Cl, F or Br.
Embodiment 12. A compound of Embodiment 11 wherein R⁴ is Cl or F.
Embodiment 13. A compound of Embodiment 12 wherein R⁴ is Cl.
Embodiment 14. A compound of Formula 1 or any one of Embodiments 1 through 13 wherein n is 0 or 1.
Embodiment 15. A compound of Embodiment 14 wherein n is 1.
Embodiment 16. A compound of Embodiment 14 wherein n is 0.

Embodiments of this invention, including Embodiments 1-16 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-16 above as well as any other embodiments described herein, and any combination thereof, pertain to the compositions and methods of the present invention.

Combinations of Embodiments 1-16 are illustrated by:

Embodiment A. A compound of Formula 1 wherein
each R¹ is independently halogen, cyano, nitro, C₁-C₆ alkyl, C₂-C₆ alkenyl, C₂-C₆ alkynyl, C₁-C₆ haloalkyl, C₂-C₆ alkoxyalkyl, C₂-C₆ alkylcarbonyl, C₂-C₆ alkoxycarbonyl, C₂-C₆ alkylaminocarbonyl, C₃-C₈ dialkylaminocarbonyl, C₂-C₆ cyanoalkyl, C₁-C₆ hydroxyalkyl, C₁-C₆ alkoxy, C₁-C₆ alkylthio, C₁-C₆ alkylsulfinyl, C₁-C₆ alkylsulfonyl, C₁-C₆ alkylamino, C₂-C₆ dialkylamino or C₂-C₆ alkylcarbonylamino.

Embodiment B. A compound of Embodiment A wherein
each R¹ is independently is halogen, C₁-C₆ alkyl, C₂-C₆ alkenyl, C₁-C₆ haloalkyl, C₂-C₆ alkylcarbonyl, C₂-C₆ alkoxycarbonyl or C₁-C₆ alkoxy;
R³ is Cl, F, Br or methoxy; and
R⁴ is H, Cl, F or Br.

Embodiment C. A compound of Embodiment B wherein each R¹ is independently halogen, C₁-C₆ alkyl or C₁-C₆ alkoxy;
R³ is F or methoxy; and
R⁴ is Cl or F.

Embodiment D. A compound of Embodiment C wherein
R² is F;
R³ is F or methoxy; and
n is 0 or 1.

Embodiment E. A compound of Embodiment D wherein
R³ is attached at the para position.

Embodiment F. A compound of Embodiment E wherein
R³ is F.

Embodiment G. A compound of Embodiment E wherein
R³ is methoxy.

This invention provides a fungicidal composition comprising a compound of Formula 1 (including all geometric and stereoisomers, N-oxides, and salts thereof), and at least one other fungicide. Of note as embodiments of such compositions are compositions comprising a compound corresponding to any of the compound embodiments described above.

This invention provides a fungicidal composition comprising a compound of Formula 1 (including all geometric and stereoisomers, N-oxides, and salts thereof), and at least one additional component selected from the group consisting of surfactants, solid diluents and liquid diluents. Of note as embodiments of such compositions are compositions comprising a compound corresponding to any of the compound embodiments described above.

This invention provides a method for controlling plant diseases caused by fungal plant pathogens comprising applying to the plant or portion thereof, or to the plant seed, a fungicidally effective amount of a compound of Formula 1 (including all geometric and stereoisomers, N-oxides, and salts thereof). Of note as embodiments of such methods are methods comprising applying a fungicidally effective amount of a compound corresponding to any of the compound embodiments described above. Of particular notes are embodiments where the compounds are applied as compositions of this invention.

One or more of the following methods and variations as described in Schemes 1-6 can be used to prepare the compounds of Formula 1. The definitions of R¹, R², R³, R⁴ and n in the compounds of Formulae 1-11 below are as defined above in the Summary of the Invention unless otherwise noted.

Compounds of Formula 1 can be prepared by reacting imines of Formula 2 with phenylpropiolate derivatives of Formula 3 as shown in Scheme 1. The reaction is typically conducted in a suitable solvent, such as xylenes, toluene, chlorobenzene, and mixtures thereof. Addition of an acid-catalyst such as p-toluenesulfonic acid or a Lewis acid such as trifluoroacetic acid, aluminum chloride or aluminum bromide can facilitate the reaction. The reaction is typically conducted at a temperature between about 20 and 200° C. For representative procedures see Barluenga et al., Synthetic Communications 1983, 13, 411-417, U.S. Pat. No. 4,757,081 and European Patent 304057. Also Step C of Example 1 illustrates the method of Scheme 1.

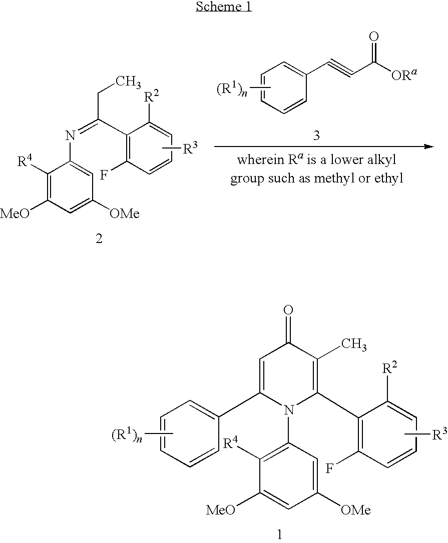

As shown below in Scheme 2, imines of Formula 2 can be prepared by reacting ketones of Formula 4 with anilines of Formula 5 under dehydrative conditions such as heating in toluene or xylenes with use of a Dean-Stark trap or in presence of molecular sieves to remove water formed in the reaction. An acid catalyst can be added to the reaction mixture to promote elimination of water. Examples of suitable acid catalyst include p-toluenesulfonic acid, acetic acid or formic acid, or a Lewis Acid such titanium tetrachloride. For representative procedures see Strekowski et al., Tetrahedron Letters 1989, 30(39), 5197-5160; Rhee et al., Synlett 2003, (1), 112-114 and U.S. Pat. No. 1,938,890. Also, Step B of Example 1 illustrates the preparation of a compound of Formula 2.

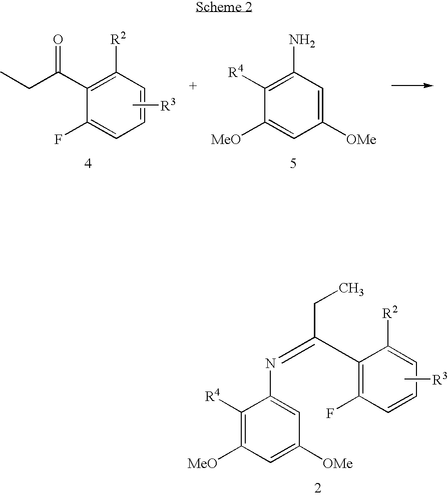

Compounds of Formula 3 can be prepared by a number of methods known in the art. According to the method of Scheme 3, an alkynylzinc intermediate is first generated in situ by treatment of an alkyne of Formula 6 with lithium diisopropylamide (LDA) followed by zinc bromide or zinc chloride. Treatment of the alkynylzinc intermediate with a phenyl halide of Formula 7 in the presence of a palladium catalyst such as tetrakis(triphenylphosphine)palladium (Pd(PPd₃)₄) and a solvent such a tetrahydrofuran at a temperature between about −78° C. and room temperature provides the corresponding compound of Formula 3. For references illustrating this procedure and related methods see Anastasia et al., Organic Letters 2001, 3, 3111-3113; King et al., Journal of the Chemical Society., Chemical Communications 1977, (19), 683-684; and King et al., Journal of Organic Chemistry 1978, 43(2), 358-360. Also, Step A of Example 1 illustrates the preparation of a compound of Formula 3 by the method of Scheme 3.

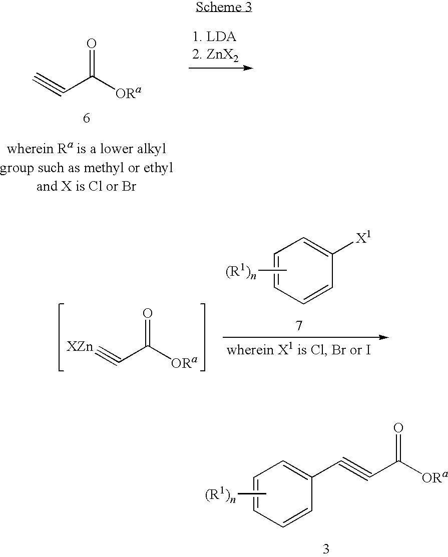

Alternatively, compounds of Formula 3 can be prepared via a cross-coupling reaction of phenylboronic acids and alkynes according to the procedure by Zou et al., Tetrahedron Letters 2003, 44, 8709-8711.

Anilines of Formula 5 are commercially available and can be readily synthesized by methods known in the art; see, for example, Sun et al., Journal of Organic Chemistry 1997, 62, 6469-6475 and PCT Patent Publication WO 2003/064398.

Compounds of Formula 4 can be conveniently prepared via a Friedel-Crafts acylation reaction as shown in Scheme 4. In this method a compound of Formula 8 is reacted with propionyl chloride in the presence of aluminum chloride. The reaction can be run in a solvent such methylene chloride or chloroform, or without solvent other than the compound of Formula 8 and propionyl chloride. The reaction is typically carried out at a temperature between about 0° C. and the reflux temperature of the reaction mixture. For representative procedures see Joshi et al., Journal of Fluorine Chemistry 1980, 15, 245-52; Watanabe et al., Bioorganic & Medicinal Chemistry Letters 2008, 185, 1478-1483 and Xu et al., Synthetic Communications 2005, 35, 2345-2353.

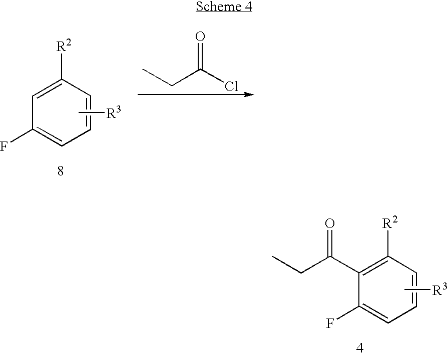

Alternatively, compounds of Formula 4 can be prepared as shown in Scheme 5. In this method an organometallic reagent of Formula 9 wherein M is MgX², Li or ZnX² and X² is Cl, Br or I is contacted with an electrophile of Formula 10 wherein Lg is a leaving group such as halogen, resulting in the introduction of a propionyl group onto Formula 9 to provide a compound of Formula 4.

Some organometallic reagents of Formula 9 are commercially available (e.g., phenyl Grignard reagents); also many known methods in the art for preparing organometallic reagents involving metallation with a metalating agent such as n-butyllithium (n-BuLi), lithium diisopropylamide (LDA) or a Grignard reagent (e.g., EtMgBr) can be used to prepare compounds of Formula 9. For representative procedures see Watanabe et al., Bioorganic & Medicinal Chemistry Letters 2008, 185, 1478-1483.

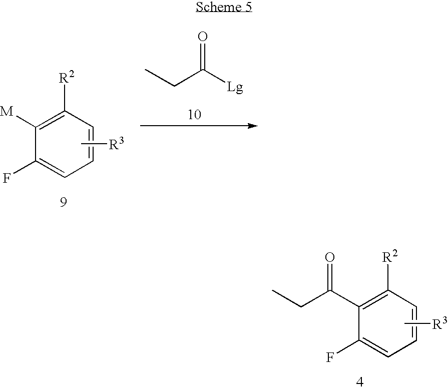

Compounds of Formula 1 can also be prepared by condensation of triketones of Formula 11 with anilines of Formula 5 as shown in Scheme 6. Typically the reaction is run in a solvent such as toluene, xylenes, chlorobenzene or N,N-dimethylformamide, at a temperature between about room temperature and 200° C. An acid catalyst can be added to the reaction mixture to promote elimination of water. Examples of suitable acid catalysts include, sulfuric acid, hydrochloric acid, p-toluenesulfonic acid, acetic acid, formic acid, or a Lewis acid such titanium tetrachloride or aluminum chloride. Use of a Dean-Stark trap or molecular sieves can be used to remove water formed in the reaction. For representative references describing the method of Scheme 5 see U.S. Pat. No. 4,757,081 and European Patent 304057.

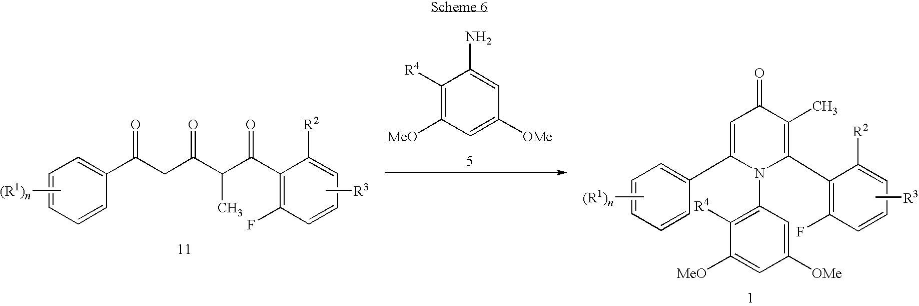

Compounds of Formula 11 can be prepared by a number of procedures. For a particularly useful procedure see Japanese Patent Application Publication 03120262.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “d” means doublet, “t” means triplet, “q” means quartet, “m” means multiplet.

### Example 1

### Preparation of 1-(2-chloro-3-5-dimethoxyphenyl)-6-(4-fluorophenyl)-3-methyl-2-(2,4,6-trifluorophenyl)-4-(1H)-pyridinone

### Step A: Preparation of ethyl 4-fluorophenylpropynoate

To a stirred mixture of diisopropylamine (6.20 g, 61.3 mmol) in tetrahydrofuran at −78° C. under an argon atmosphere (15 mL), n-butyllithium (3.92 g, 61.2 mmol, 1.6 M in hexanes) was added. The reaction mixture was maintained under an argon atmosphere and stirred for 20 minutes at −50° C. The reaction mixture was cooled to −78° C., and a solution of ethyl propiolate (5.0 g, 51 mmol) in tetrahydrofuran (15 mL) was added. After stirring for 20 minutes at −78° C., a mixture of zinc chloride (8.34 g, 61.2 mmol) in tetrahydrofuran (20 mL) was added to the reaction mixture and stirring was continued at −78° C.

In another reaction vessel, tetrakis(triphenylphosphine)palladium (Pd(PPd₃)₄) (2.9 g, 2.5 mmol) was added to a mixture of 1-fluoro-4-iodobenzene (11.32 g, 51.0 mmol) in tetrahydrofuran (25 mL). The reaction mixture was maintained under an argon atmosphere, stirred for 15 minutes, then cooled to −78° C., and the reaction mixture formed from ethyl 4-fluorophenylpropynoate was added via syringe. After the addition was complete, the reaction mixture was allowed to slowly warm to room temperature. After 1.3 h, saturated aqueous ammonium chloride solution was added to the reaction mixture, and the mixture was extracted with ethyl acetate. The organic layer was washed with water and saturated aqueous sodium chloride solution, dried (Na₂SO₄), filtered and concentrated. The resulting residue was purified by column chromatography on silica gel (using 2% ethyl acetate-petroleum ether as eluant) to provide the title compound as a pale yellow solid (5 g).

¹H NMR (CDCl₃): δ 6.74-6.67 (m, 2H), 2.87 (q, J=7.2 Hz, 2H), 1.19 (t, J=7.2 Hz, 3H).

### Step B: Preparation of 2-chloro-3,5-dimethoxy-N-[1-(2,4,6-trifluorophenyl)propylidene]benzenamine

A mixture of 1-(2,4,6-trifluorophenyl)-1-propanone (7.0 g, 37.2 mmol), 2-chloro-3,5-dimethoxybenzenamine (see European Patent 1333028 for a method of preparation) (6.96 g, 37.1 mmol) and p-toluenesulfonic acid (1.41 g, 8.2 mmol) in toluene (80 mL) was heated at reflux with use of a Dean-Stark trap to remove water. After 20 h, the reaction mixture was cooled to room temperature and diluted with ethyl acetate. The organic layer was washed with water, sulfuric acid (1 N), water and saturated aqueous sodium chloride solution. The organic layer was dried (Na₂SO₄), filtered and concentrated to provide the title compound (10 g) which was used without further purification in Step C below.

### Step C: Preparation of 1-(2-chloro-3-5-dimethoxyphenyl)-6-(4-fluorophenyl)-3-methyl-2-(2,4,6-trifluorophenyl)-4-(1H)-pyridinone

A mixture of ethyl 4-fluorophenylpropynoate (i.e. the product of Step A) (2.68 g, 14.0 mmol), 2-chloro-3,5-dimethoxy-N-[1-(2,4,6-trifluorophenyl)propylidene]benzenamine (i.e. the product of Step B) (5.0 g, 13.9 mmol) and aluminum chloride (2.79 g, 21.1 mmol) in toluene (160 mL) was heated at reflux for 16 h. The reaction mixture was cooled, and poured onto ice, and the mixture was extracted with chloroform. The chloroform layer was washed with sulfuric acid (1 N), sodium hydroxide solution (5% in water), dried (Na₂SO₄), filtered and concentrated. The resulting residue (7 g) was purified by column chromatography to provide a solid which was further purified by reverse phase HPLC to provide the title compound, a compound of the present invention, as an off-white solid (65 mg).

¹H NMR (CDCl₃): δ 7.24-7.23 (m, 2H), 6.88 (t, J=8.6 Hz, 2H), 6.62-6.56 (m, 3H), 6.38-6.37 (m, 1H), 6.23 (d, J=2.4 Hz, 1H), 3.7 (s, 3H), 3.65 (s, 3H), 1.90 (s, 3H).

By the procedures described herein together with methods known in the art, the following compounds of Table 1 can be prepared. The following abbreviations are used in the Tables which follow: c means cyclo, Me means methyl, Pr means propyl, c-Pr means cyclopropyl, OMe means methoxy, SMe means methylthio, CN means cyano, means phenyl, NO₂ means nitro, S(O)Me means methylsulfinyl, and S(O)₂Me means methylsulfonyl. In the following table a dash (“-”) in the (R¹)ₙ indicates n is 0 and hydrogen is present at all available positions.

TABLE 1

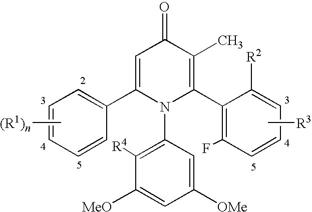

| R² is F, R³ is | R² is F, R³ is | R² is F, R³ is | R² is F, R³ is |
|---|---|---|---|
| 4-F and R⁴ | 4-F and R⁴ | 4-OMe and R⁴ | 4-OMe and R⁴ |
| is H. | is Cl. | is H. | is Cl. |
| (R¹)ₙ | (R¹)ₙ | (R¹)ₙ | (R¹)ₙ |
| — | — | — | — |
| 2-F | 2-F | 2-F | 2-F |
| 3-F | 3-F | 3-F | 3-F |
| 4-F | 4-F | 4-F | 4-F |
| 2-Cl | 2-Cl | 2-Cl | 2-Cl |
| 3-Cl | 3-Cl | 3-Cl | 3-Cl |
| 4-Cl | 4-Cl | 4-Cl | 4-Cl |
| 2-Br | 2-Br | 2-Br | 2-Br |
| 3-Br | 3-Br | 3-Br | 3-Br |
| 4-Br | 4-Br | 4-Br | 4-Br |
| 2-I | 2-I | 2-I | 2-I |
| 3-I | 3-I | 3-I | 3-I |
| 4-I | 4-I | 4-I | 4-I |
| 2,4-di-F | 2,4-di-F | 2,4-di-F | 2,4-di-F |
| 2-F, 4-Cl | 2-F, 4-Cl | 2-F, 4-Cl | 2-F, 4-Cl |
| 2-Cl, 4-F | 2-Cl, 4-F | 2-Cl, 4-F | 2-Cl, 4-F |
| 2-CF₃ | 2-CF₃ | 2-CF₃ | 2-CF₃ |
| 4-CF₃ | 4-CF₃ | 4-CF₃ | 4-CF₃ |
| 2-F, 4-CF₃ | 2-F, 4-CF₃ | 2-F, 4-CF₃ | 2-F, 4-CF₃ |
| 2-Me | 2-Me | 2-Me | 2-Me |
| 4-Me | 4-Me | 4-Me | 4-Me |
| 2-OMe | 2-OMe | 2-OMe | 2-OMe |
| 4-OMe | 4-OMe | 4-OMe | 4-OMe |
| 2-SMe | 2-SMe | 2-SMe | 2-SMe |
| 4-SMe | 4-SMe | 4-SMe | 4-SMe |
| 4-SCF₃ | 4-SCF₃ | 4-SCF₃ | 4-SCF₃ |
| 2-S(O)Me | 2-S(O)Me | 2-S(O)Me | 2-S(O)Me |
| 4-S(O)Me | 4-S(O)Me | 4-S(O)Me | 4-S(O)Me |
| 4-S(O)₂Me | 4-S(O)₂Me | 4-S(O)₂Me | 4-S(O)₂Me |
| 2-CN | 2-CN | 2-CN | 2-CN |
| 3-CN | 3-CN | 3-CN | 3-CN |
| 4-CN | 4-CN | 4-CN | 4-CN |
| 2-F, 4-CN | 2-F, 4-CN | 2-F, 4-CN | 2-F, 4-CN |
| 4-c-Pr | 4-c-Pr | 4-c-Pr | 4-c-Pr |
| 2-C(═O)Me | 2-C(═O)Me | 2-C(═O)Me | 2-C(═O)Me |
| 4-CO₂Me | 4-CO₂Me | 4-CO₂Me | 4-CO₂Me |
| 3-OCH₂OMe | 3-OCH₂OMe | 3-OCH₂OMe | 3-OCH₂OMe |
| 4-C(═O)NMe₂ | 4-C(═O)NMe₂ | 4-C(═O)NMe₂ | 4-C(═O)NMe₂ |
| 4-S(O)₂NMe₂ | 4-S(O)₂NMe₂ | 4-S(O)₂NMe₂ | 4-S(O)₂NMe₂ |
| 3-NO₂ | 3-NO₂ | 3-NO₂ | 3-NO₂ |
| R² is Cl, R³ is | R² is Cl, R³ is | R² is Cl, R³ is | R² is Cl, R³ is |
| 4-F and R⁴ | 4-F and R⁴ | 4-OMe and R⁴ | 4-OMe and R⁴ |
| is H. | is Cl. | is H. | is Cl. |
| (R¹)ₙ | (R¹)ₙ | (R¹)ₙ | (R¹)ₙ |
| — | — | — | — |
| 2-F | 2-F | 2-F | 2-F |
| 3-F | 3-F | 3-F | 3-F |
| 4-F | 4-F | 4-F | 4-F |
| 2-Cl | 2-Cl | 2-Cl | 2-Cl |
| 3-Cl | 3-Cl | 3-Cl | 3-Cl |
| 4-Cl | 4-Cl | 4-Cl | 4-Cl |
| 2-Br | 2-Br | 2-Br | 2-Br |
| 3-Br | 3-Br | 3-Br | 3-Br |
| 4-Br | 4-Br | 4-Br | 4-Br |
| 2-I | 2-I | 2-I | 2-I |
| 3-I | 3-I | 3-I | 3-I |
| 4-I | 4-I | 4-I | 4-I |
| 2,4-di-F | 2,4-di-F | 2,4-di-F | 2,4-di-F |
| 2-F, 4-Cl | 2-F, 4-Cl | 2-F, 4-Cl | 2-F, 4-Cl |
| 2-Cl, 4-F | 2-Cl, 4-F | 2-Cl, 4-F | 2-Cl, 4-F |
| 2-CF₃ | 2-CF₃ | 2-CF₃ | 2-CF₃ |
| 4-CF₃ | 4-CF₃ | 4-CF₃ | 4-CF₃ |
| 2-F, 4-CF₃ | 2-F, 4-CF₃ | 2-F, 4-CF₃ | 2-F, 4-CF₃ |
| 2-Me | 2-Me | 2-Me | 2-Me |
| 4-Me | 4-Me | 4-Me | 4-Me |
| 2-OMe | 2-OMe | 2-OMe | 2-OMe |
| 4-OMe | 4-OMe | 4-OMe | 4-OMe |
| 2-SMe | 2-SMe | 2-SMe | 2-SMe |
| 4-SMe | 4-SMe | 4-SMe | 4-SMe |
| 4-SCF₃ | 4-SCF₃ | 4-SCF₃ | 4-SCF₃ |
| 2-S(O)Me | 2-S(O)Me | 2-S(O)Me | 2-S(O)Me |
| 4-S(O)Me | 4-S(O)Me | 4-S(O)Me | 4-S(O)Me |
| 4-S(O)₂Me | 4-S(O)₂Me | 4-S(O)₂Me | 4-S(O)₂Me |
| 2-CN | 2-CN | 2-CN | 2-CN |
| 3-CN | 3-CN | 3-CN | 3-CN |
| 4-CN | 4-CN | 4-CN | 4-CN |
| 2-F, 4-CN | 2-F, 4-CN | 2-F, 4-CN | 2-F, 4-CN |
| 4-c-Pr | 4-c-Pr | 4-c-Pr | 4-c-Pr |
| 2-C(═O)Me | 2-C(═O)Me | 2-C(═O)Me | 2-C(═O)Me |
| 4-CO₂Me | 4-CO₂Me | 4-CO₂Me | 4-CO₂Me |
| 3-OCH₂OMe | 3-OCH₂OMe | 3-OCH₂OMe | 3-OCH₂OMe |
| 4-C(═O)NMe₂ | 4-C(═O)NMe₂ | 4-C(═O)NMe₂ | 4-C(═O)NMe₂ |
| 4-S(O)₂NMe₂ | 4-S(O)₂NMe₂ | 4-S(O)₂NMe₂ | 4-S(O)₂NMe₂ |
| 3-NO₂ | 3-NO₂ | 3-NO₂ | 3-NO₂ |
| R² is F, R³ is | R² is F, R³ is | R² is F, R³ is | R² is F, R³ is |
| 3-F and R⁴ | 3-F and R⁴ | 4-Cl and R⁴ | 4-Cl and R⁴ |
| is H. | is Cl. | is H. | is Cl. |
| (R¹)ₙ | (R¹)ₙ | (R¹)ₙ | (R¹)ₙ |
| 2-F | 2-F | 2-F | 2-F |
| 3-F | 3-F | 3-F | 3-F |
| 4-F | 4-F | 4-F | 4-F |
| 2-Cl | 2-Cl | 2-Cl | 2-Cl |
| 3-Cl | 3-Cl | 3-Cl | 3-Cl |
| 4-Cl | 4-Cl | 4-Cl | 4-Cl |
| 2-Br | 2-Br | 2-Br | 2-Br |
| 3-Br | 3-Br | 3-Br | 3-Br |
| 4-Br | 4-Br | 4-Br | 4-Br |
| 2-I | 2-I | 2-I | 2-I |
| 3-I | 3-I | 3-I | 3-I |
| 4-I | 4-I | 4-I | 4-I |
| 2,4-di-F | 2,4-di-F | 2,4-di-F | 2,4-di-F |
| 2-F, 4-Cl | 2-F, 4-Cl | 2-F, 4-Cl | 2-F, 4-Cl |
| 2-Cl, 4-F | 2-Cl, 4-F | 2-Cl, 4-F | 2-Cl, 4-F |
| 2-CF₃ | 2-CF₃ | 2-CF₃ | 2-CF₃ |
| 4-CF₃ | 4-CF₃ | 4-CF₃ | 4-CF₃ |
| 2-F, 4-CF₃ | 2-F, 4-CF₃ | 2-F, 4-CF₃ | 2-F, 4-CF₃ |
| 2-Me | 2-Me | 2-Me | 2-Me |
| 4-Me | 4-Me | 4-Me | 4-Me |
| 2-OMe | 2-OMe | 2-OMe | 2-OMe |
| 4-OMe | 4-OMe | 4-OMe | 4-OMe |
| 2-SMe | 2-SMe | 2-SMe | 2-SMe |
| 4-SMe | 4-SMe | 4-SMe | 4-SMe |
| 4-SCF₃ | 4-SCF₃ | 4-SCF₃ | 4-SCF₃ |
| 2-S(O)Me | 2-S(O)Me | 2-S(O)Me | 2-S(O)Me |
| 4-S(O)Me | 4-S(O)Me | 4-S(O)Me | 4-S(O)Me |
| 4-S(O)₂Me | 4-S(O)₂Me | 4-S(O)₂Me | 4-S(O)₂Me |
| 2-CN | 2-CN | 2-CN | 2-CN |
| 3-CN | 3-CN | 3-CN | 3-CN |
| 4-CN | 4-CN | 4-CN | 4-CN |
| 2-F, 4-CN | 2-F, 4-CN | 2-F, 4-CN | 2-F, 4-CN |
| 4-c-Pr | 4-c-Pr | 4-c-Pr | 4-c-Pr |
| 2-C(═O)Me | 2-C(═O)Me | 2-C(═O)Me | 2-C(═O)Me |
| 4-CO₂Me | 4-CO₂Me | 4-CO₂Me | 4-CO₂Me |
| 3-OCH₂OMe | 3-OCH₂OMe | 3-OCH₂OMe | 3-OCH₂OMe |
| 4-C(═O)NMe₂ | 4-C(═O)NMe₂ | 4-C(═O)NMe₂ | 4-C(═O)NMe₂ |
| 4-S(O)₂NMe₂ | 4-S(O)₂NMe₂ | 4-S(O)₂NMe₂ | 4-S(O)₂NMe₂ |
| 3-NO₂ | 3-NO₂ | 3-NO₂ | 3-NO₂ |
| R² is Cl, R³ is | R² is Cl, R³ is | R² is Cl, R³ is | R² is Cl, R³ is |
| 3-F and R⁴ | 3-F and R⁴ | 4-Cl; R⁴ | 4-Cl and R⁴ |
| is H. | is Cl. | is H. | is Cl. |
| (R¹)ₙ | (R¹)ₙ | (R¹)ₙ | (R¹)ₙ |
| — | — | — | — |
| 2-F | 2-F | 2-F | 2-F |
| 3-F | 3-F | 3-F | 3-F |
| 4-F | 4-F | 4-F | 4-F |
| 2-Cl | 2-Cl | 2-Cl | 2-Cl |
| 3-Cl | 3-Cl | 3-Cl | 3-Cl |
| 4-Cl | 4-Cl | 4-Cl | 4-Cl |
| 2-Br | 2-Br | 2-Br | 2-Br |
| 3-Br | 3-Br | 3-Br | 3-Br |
| 4-Br | 4-Br | 4-Br | 4-Br |
| 2-I | 2-I | 2-I | 2-I |
| 3-I | 3-I | 3-I | 3-I |
| 4-I | 4-I | 4-I | 4-I |
| 2,4-di-F | 2,4-di-F | 2,4-di-F | 2,4-di-F |
| 2-F, 4-Cl | 2-F, 4-Cl | 2-F, 4-Cl | 2-F, 4-Cl |
| 2-Cl, 4-F | 2-Cl, 4-F | 2-Cl, 4-F | 2-Cl, 4-F |
| 2-CF₃ | 2-CF₃ | 2-CF₃ | 2-CF₃ |
| 4-CF₃ | 4-CF₃ | 4-CF₃ | 4-CF₃ |
| 2-F, 4-CF₃ | 2-F, 4-CF₃ | 2-F, 4-CF₃ | 2-F, 4-CF₃ |
| 2-Me | 2-Me | 2-Me | 2-Me |
| 4-Me | 4-Me | 4-Me | 4-Me |
| 2-OMe | 2-OMe | 2-OMe | 2-OMe |
| R² is F, R³ is | R² is F, R³ is | R² is F, R³ is | R² is F, R³ is |
| 3-Br and R⁴ | 3-Br and R⁴ | 4-Br and R⁴ | 4-Br and R⁴ |
| is H. | is Cl. | is H. | is Cl. |
| (R¹)ₙ | (R¹)ₙ | (R¹)ₙ | (R¹)ₙ |
| 2-F | 2-F | 2-F | 2-F |
| 3-F | 3-F | 3-F | 3-F |
| 4-F | 4-F | 4-F | 4-F |
| 2-Cl | 2-Cl | 2-Cl | 2-Cl |
| 3-Cl | 3-Cl | 3-Cl | 3-Cl |
| 4-Cl | 4-Cl | 4-Cl | 4-Cl |
| 2-Br | 2-Br | 2-Br | 2-Br |
| 3-Br | 3-Br | 3-Br | 3-Br |
| 4-Br | 4-Br | 4-Br | 4-Br |
| 2-I | 2-I | 2-I | 2-I |
| 3-I | 3-I | 3-I | 3-I |
| 4-I | 4-I | 4-I | 4-I |
| 2,4-di-F | 2,4-di-F | 2,4-di-F | 2,4-di-F |
| 2-F, 4-Cl | 2-F, 4-Cl | 2-F, 4-Cl | 2-F, 4-Cl |
| 2-Cl, 4-F | 2-Cl, 4-F | 2-Cl, 4-F | 2-Cl, 4-F |
| 2-CF₃ | 2-CF₃ | 2-CF₃ | 2-CF₃ |
| 4-CF₃ | 4-CF₃ | 4-CF₃ | 4-CF₃ |
| 2-F, 4-CF₃ | 2-F, 4-CF₃ | 2-F, 4-CF₃ | 2-F, 4-CF₃ |
| 2-Me | 2-Me | 2-Me | 2-Me |
| 4-Me | 4-Me | 4-Me | 4-Me |
| 2-OMe | 2-OMe | 2-OMe | 2-OMe |
| R² is Cl, R³ is | R² is Cl, R³ is | R² is Cl, R³ is | R² is Cl, R³ is |
| 5-F and R⁴ | 5-F and R⁴ | 5-Cl and R⁴ | 5-Cl and R⁴ |
| is H. | is Cl. | is H. | is Cl. |
| (R¹)ₙ | (R¹)ₙ | (R¹)ₙ | (R¹)ₙ |
| 2-F | 2-F | 2-F | 2-F |
| 3-F | 3-F | 3-F | 3-F |
| 4-F | 4-F | 4-F | 4-F |
| 2-Cl | 2-Cl | 2-Cl | 2-Cl |
| 3-Cl | 3-Cl | 3-Cl | 3-Cl |
| 4-Cl | 4-Cl | 4-Cl | 4-Cl |
| 2-Br | 2-Br | 2-Br | 2-Br |
| 3-Br | 3-Br | 3-Br | 3-Br |
| 4-Br | 4-Br | 4-Br | 4-Br |
| 2-I | 2-I | 2-I | 2-I |
| 3-I | 3-I | 3-I | 3-I |
| 4-I | 4-I | 4-I | 4-I |
| 2,4-di-F | 2,4-di-F | 2,4-di-F | 2,4-di-F |
| 2-F, 4-Cl | 2-F, 4-Cl | 2-F, 4-Cl | 2-F, 4-Cl |
| 2-Cl, 4-F | 2-Cl, 4-F | 2-Cl, 4-F | 2-Cl, 4-F |
| 2-CF₃ | 2-CF₃ | 2-CF₃ | 2-CF₃ |
| 4-CF₃ | 4-CF₃ | 4-CF₃ | 4-CF₃ |
| 2-F, 4-CF₃ | 2-F, 4-CF₃ | 2-F, 4-CF₃ | 2-F, 4-CF₃ |
| 2-Me | 2-Me | 2-Me | 2-Me |
| 4-Me | 4-Me | 4-Me | 4-Me |
| 2-OMe | 2-OMe | 2-OMe | 2-OMe |

### Formulation/Utility

A compound of Formula 1 of this invention will generally be used as a fungicidal active ingredient in a composition, i.e. formulation, with at least one additional component selected from the group consisting of surfactants, solid diluents and liquid diluents, which serve as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, pills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto vegetable seeds as seed treatments before planting to protect developing roots and other subterranean plant parts and/or foliage through systemic uptake.

The formulations will typically contain effective amounts of active ingredient, diluent and surfactant within the following approximate ranges in the Formulation Table which add up to 100 percent by weight.

| | FORMULATION TABLE | | |
|---|---|---|---|
| | Weight Percent | | |
| | Active | | |
| | Ingredient | Diluent | Surfactant |
| Water-Dispersible and Water-soluble | 0.001-90 | 0-99.999 | 0-15 |
| Granules, Tablets and Powders. | | | |
| Oil Dispersions, Suspensions, | 1-50 | 40-99 | 0-50 |
| Emulsions, Solutions (including | | | |
| Emulsifiable Concentrates) | | | |
| Dusts | 1-25 | 70-99 | 0-5 |
| Granules and Pellets | 0.001-95 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, triacetin, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, acetate and isobornyl acetate, other esters such as alkylated lactate esters, dibasic esters and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids. Such formulation auxiliaries and additives may control: pH (buffers), foaming during processing (antifoams such polyorganosiloxanes (e.g., Rhodorsil® 416)), sedimentation of active ingredients (suspending agents), viscosity (thixotropic thickeners), in-container microbial growth (antimicrobials), product freezing (antifreezes), color (dyes/pigment dispersions (e.g., Pro-Ized® Colorant Red)), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and, usually, grinding as in a hammer mill or fluid-energy mill. Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, UK, 2000.

In the following Examples, all percentages are by weight and all formulations are prepared in conventional ways. Compound numbers refer to compounds in Index Table A.

### Example A

High Strength Concentrate

| | |
|---|---|
| Compound 1 | 98.5% |
| silica aerogel | 0.5% |
| synthetic amorphous fine silica | 1.0% |

### Example B

Wettable Powder

| | |
|---|---|
| Compound 2 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

Granule

| | |
|---|---|
| Compound 3 | 10.0% |
| attapulgite granules (low volatile matter, | 90.0% |
| 0.71/0.30 mm; U.S.S. No. 25-50 sieves) | |

### Example D

Aqueous Suspension

| | |
|---|---|
| Compound 4 | 25.0% |
| hydrated attapulgite | 3.0% |
| crude calcium ligninsulfonate | 10.0% |
| sodium dihydrogen phosphate | 0.5% |
| water | 61.5.0% |

### Example E

Extruded Pellet

| | |
|---|---|
| Compound 5 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example F

Microemulsion

| | |
|---|---|
| Compound 1 | 5.0% |
| polyvinylpyrrolidone-vinyl acetate copolymer | 30.0% |
| C₈-C₁₀ alkylpolyglycoside | 30.0% |
| glyceryl monooleate | 15.0% |
| Water | 20.0% |

### Example G

Emulsifiable Concentrate

| | |
|---|---|
| Compound 2 | 10.0% |
| polyoxyethylene sorbitol hexoleate | 20.0% |
| C₆-C₁₀ fatty acid methyl ester | 70.0% |

Formulations such as those in the Formulation Table above are typically diluted with water to form aqueous compositions suitable for convenient application. Aqueous compositions for direct applications to the plant or portion thereof (e.g., spray tank compositions) typically at least about 1 ppm or more (e.g., from 1 ppm to 300 ppm) of the compound(s) of this invention.

The compounds of this invention are useful as plant disease control agents. The present invention therefore further comprises a method for controlling plant diseases caused by fungal plant pathogens comprising applying to the plant or portion thereof to be protected, or to the plant seed to be protected, an effective amount of a compound of the invention or a fungicidal composition containing said compound. The compounds and/or compositions of this invention provide control of diseases caused by a broad spectrum of fungal plant pathogens in the Basidiomycete, Ascomycete, Oomycete and Deuteromycete classes. They are effective in controlling a broad spectrum of plant diseases, particularly foliar pathogens of ornamental, turf, vegetable, field, cereal, and fruit crops. These pathogens include: Oomycetes, including Phytophthora diseases such as Phytophthora infestans, Phytophthora megasperma, Phytophthora parasitica, Phytophthora cinnamomi and Phytophthora capsici, Pythium diseases such as Pythium aphanidermatum, and diseases in the Peronosporaceae family such as Plasmopara viticola, Peronospora spp. (including Peronospora tabacina and Peronospora parasitica), Pseudoperonospora spp. (including Pseudoperonospora cubensis) and Bremia lactucae; Ascomycetes, including Alternaria diseases such as Alternaria solani and Alternaria brassicae, Guignardia diseases such as Guignardia bidwell, Venturia diseases such as Venturia inaequalis, Septoria diseases such as Septoria nodorum and Septoria tritici, powdery mildew diseases such as Erysiphe spp. (including Erysiphe graminis and Erysiphe polygoni), Uncinula necatur, Sphaerotheca fuligena and Podosphaera leucotricha, Pseudocercosporella herpotrichoides, Botrytis diseases such as Botrytis cinerea, Monilinia fructicola, Sclerotinia diseases such as Sclerotinia sclerotiorum, Magnaporthe grisea, Phomopsis viticola, Helminthosporium diseases such as Helminthosporium tritici repentis, Pyrenophora teres, anthracnose diseases such as Glomerella or Colletotrichum spp. (such as Colletotrichum graminicola and Colletotrichum orbiculare), and Gaeumannomyces graminis; Basidiomycetes, including rust diseases caused by Puccinia spp. (such as Puccinia recondite, Puccinia striiformis, Puccinia hordei, Puccinia graminis and Puccinia arachidis), Hemileia vastatrix and Phakopsora pachyrhizi; other pathogens including Rhizoctonia spp. (such as Rhizoctonia solani); Fusarium diseases such as Fusarium roseum, Fusarium graminearum and Fusarium oxysporum; Verticillium dahliae; Sclerotium rolfsii; Rynchosporium secalis; Cercosporidium personatum, Cercospora arachidicola and Cercospora beticola; and other genera and species closely related to these pathogens. In addition to their fungicidal activity, the compositions or combinations also have activity against bacteria such as Erwinia amylovora, Xanthomonas campestris, Pseudomonas syringae, and other related species.

Plant disease control is ordinarily accomplished by applying an effective amount of a compound of this invention either pre- or post-infection, to the portion of the plant to be protected such as the roots, stems, foliage, fruit, seeds, tubers or bulbs, or to the media (soil or sand) in which the plants to be protected are growing. The compounds can also be applied to seeds to protect the seeds and seedlings developing from the seeds. The compounds can also be applied through irrigation water to treat plants.

Rates of application for these compounds can be influenced by many factors of the environment and should be determined under actual use conditions. Foliage can normally be protected when treated at a rate of from less than about 1 g/ha to about 5,000 g/ha of active ingredient. Seed and seedlings can normally be protected when seed is treated at a rate of from about 0.1 to about 10 g per kilogram of seed.

Compounds of this invention can also be mixed with one or more other biologically active compounds or agents including fungicides, insecticides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Thus the present invention also pertains to a composition comprising a fungicidally effective amount of a compound of Formula 1 and a biologically effective amount of at least one additional biologically active compound or agent and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

Of note is a composition which in addition to the compound of Formula 1 include at least one fungicidal compound selected from the group consisting of the classes (1) methyl benzimidazole carbamate (MBC) fungicides; (2) dicarboximide fungicides; (3) demethylation inhibitor (DMI) fungicides; (4) phenylamide fungicides; (5) amine/morpholine fungicides; (6) phospholipid biosynthesis inhibitor fungicides; (7) carboxamide fungicides; (8) hydroxy(2-amino-)pyrimidine fungicides; (9) anilinopyrimidine fungicides; (10) N-phenyl carbamate fungicides; (11) quinone outside inhibitor (QoI) fungicides; (12) phenylpyrrole fungicides; (13) quinoline fungicides; (14) lipid peroxidation inhibitor fungicides; (15) melanin biosynthesis inhibitors-reductase (MBI-R) fungicides; (16) melanin biosynthesis inhibitors-dehydratase (MBI-D) fungicides; (17) hydroxyanilide fungicides; (18) squalene-epoxidase inhibitor fungicides; (19) polyoxin fungicides; (20) phenylurea fungicides; (21) quinone inside inhibitor (QiI) fungicides; (22) benzamide fungicides; (23) enopyranuronic acid antibiotic fungicides; (24) hexopyranosyl antibiotic fungicides; (25) glucopyranosyl antibiotic: protein synthesis fungicides; (26) glucopyranosyl antibiotic: trehalase and inositol biosynthesis fungicides; (27) cyanoacetamideoxime fungicides; (28) carbamate fungicides; (29) oxidative phosphorylation uncoupling fungicides; (30) organo tin fungicides; (31) carboxylic acid fungicides; (32) heteroaromatic fungicides; (33) phosphonate fungicides; (34) phthalamic acid fungicides; (35) benzotriazine fungicides; (36) benzene-sulfonamide fungicides; (37) pyridazinone fungicides; (38) thiophene-carboxamide fungicides; (39) pyrimidinamide fungicides; (40) carboxylic acid amide (CAA) fungicides; (41) tetracycline antibiotic fungicides; (42) thiocarbamate fungicides; (43) benzamide fungicides; (44) host plant defense induction fungicides; (45) multi-site contact activity fungicides; (46) fungicides other than classes (1) through (45); and salts of compounds of classes (1) through (46).

Further descriptions of these classes of fungicidal compounds are provided below.

(1) “Methyl benzimidazole carbamate (MBC) fungicides” (Fungicide Resistance Action Committee (FRAC) code 1) inhibit mitosis by binding to β-tubulin during microtubule assembly. Inhibition of microtubule assembly can disrupt cell division, transport within the cell and cell structure. Methyl benzimidazole carbamate fungicides include benzimidazole and thiophanate fungicides. The benzimidazoles include benomyl, carbendazim, fuberidazole and thiabendazole. The thiophanates include thiophanate and thiophanate-methyl.

(2) “Dicarboximide fungicides” (Fungicide Resistance Action Committee (FRAC) code 2) are proposed to inhibit a lipid peroxidation in fungi through interference with NADH cytochrome c reductase. Examples include chlozolinate, iprodione, procymidone and vinclozolin.

(3) “Demethylation inhibitor (DMI) fungicides” (Fungicide Resistance Action Committee (FRAC) code 3) inhibit Cl4-demethylase, which plays a role in sterol production. Sterols, such as ergosterol, are needed for membrane structure and function, making them essential for the development of functional cell walls. Therefore, exposure to these fungicides results in abnormal growth and eventually death of sensitive fungi. DMI fungicides are divided between several chemical classes: azoles (including triazoles and imidazoles), pyrimidines, piperazines and pyridines. The triazoles include azaconazole, bitertanol, bromuconazole, cyproconazole, difenoconazole, diniconazole (including diniconazole-M), epoxiconazole, fenbuconazole, fluquinconazole, flusilazole, flutriafol, hexaconazole, imibenconazole, ipconazole, metconazole, myclobutanil, penconazole, propiconazole, prothioconazole, simeconazole, tebuconazole, tetraconazole, triadimefon, triadimenol, triticonazole and uniconazole. The imidazoles include clotrimazole, imazalil, oxpoconazole, prochloraz, pefurazoate and triflumizole. The pyrimidines include fenarimol and nuarimol. The piperazines include triforine. The pyridines include pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

(4) “Phenylamide fungicides” (Fungicide Resistance Action Committee (FRAC) code 4) are specific inhibitors of RNA polymerase in Oomycete fungi. Sensitive fungi exposed to these fungicides show a reduced capacity to incorporate uridine into rRNA. Growth and development in sensitive fungi is prevented by exposure to this class of fungicide. Phenylamide fungicides include acylalanine, oxazolidinone and butyrolactone fungicides. The acylalanines include benalaxyl, benalaxyl-M, furalaxyl, metalaxyl and metalaxyl-M/mefenoxam. The oxazolidinones include oxadixyl. The butyrolactones include ofurace.

(5) “Amine/morpholine fungicides” (Fungicide Resistance Action Committee (FRAC) code 5) inhibit two target sites within the sterol biosynthetic pathway, Δ⁸→Δ⁷ isomerase and Δ¹⁴ reductase. Sterols, such as ergosterol, are needed for membrane structure and function, making them essential for the development of functional cell walls. Therefore, exposure to these fungicides results in abnormal growth and eventually death of sensitive fungi. Amine/morpholine fungicides (also known as non-DMI sterol biosynthesis inhibitors) include morpholine, piperidine and spiroketal-amine fungicides. The morpholines include aldimorph, dodemorph, fenpropimorph, tridemorph and trimorphamide. The piperidines include fenpropidin and piperalin. The spiroketal-amines include spiroxamine.

(6) “Phospholipid biosynthesis inhibitor fungicides” (Fungicide Resistance Action Committee (FRAC) code 6) inhibit growth of fungi by affecting phospholipid biosynthesis. Phospholipid biosynthesis fungicides include phosphorothiolate and dithiolane fungicides. The phosphorothiolates include edifenphos, iprobenfos and pyrazophos. The dithiolanes include isoprothiolane.

(7) “Carboxamide fungicides” (Fungicide Resistance Action Committee (FRAC) code 7) inhibit Complex II (succinate dehydrogenase) fungal respiration by disrupting a key enzyme in the Krebs Cycle (TCA cycle) named succinate dehydrogenase. Inhibiting respiration prevents the fungus from making ATP, and thus inhibits growth and reproduction. Carboxamide fungicides include benzamides, furan carboxamides, oxathiin carboxamides, thiazole carboxamides, pyrazole carboxamides and pyridine carboxamides. The benzamides include benodanil, flutolanil and mepronil. The furan carboxamides include fenfuram. The oxathiin carboxamides include carboxin and oxycarboxin. The thiazole carboxamides include thifluzamide. The pyrazole carboxamides include furametpyr, penthiopyrad, bixafen, N-[2-(1S,2R)-[1,1′-bicyclopropyl]-2-ylphenyl]-3-(difluoromethyl)-1-methyl-1H-pyrazole-4-carboxamide and N-[2-(1,3-dimethylbutyl)phenyl]-5-fluoro-1,3-dimethyl-1H-pyrazole-4-carboxamide. The pyridine carboxamides include boscalid.

(8) “Hydroxy(2-amino-)pyrimidine fungicides” (Fungicide Resistance Action Committee (FRAC) code 8) inhibit nucleic acid synthesis by interfering with adenosine deaminase. Examples include bupirimate, dimethirimol and ethirimol.

(9) “Anilinopyrimidine fungicides” (Fungicide Resistance Action Committee (FRAC) code 9) are proposed to inhibit biosynthesis of the amino acid methionine and to disrupt the secretion of hydrolytic enzymes that lyse plant cells during infection. Examples include cyprodinil, mepanipyrim and pyrimethanil.

(10) “N-Phenyl carbamate fungicides” (Fungicide Resistance Action Committee (FRAC) code 10) inhibit mitosis by binding to β-tubulin and disrupting microtubule assembly. Inhibition of microtubule assembly can disrupt cell division, transport within the cell and cell structure. Examples include diethofencarb.

(11) “Quinone outside inhibitor (QoI) fungicides” (Fungicide Resistance Action Committee (FRAC) code 11) inhibit Complex III mitochondrial respiration in fungi by affecting ubiquinol oxidase. Oxidation of ubiquinol is blocked at the “quinone outside” (Qₒ) site of the cytochrome bc₁ complex, which is located in the inner mitochondrial membrane of fungi. Inhibiting mitochondrial respiration prevents normal fungal growth and development. Quinone outside inhibitor fungicides (also known as strobilurin fungicides) include methoxyacrylate, methoxycarbamate, oximinoacetate, oximinoacetamide, oxazolidinedione, dihydrodioxazine, imidazolinone and benzylcarbamate fungicides. The methoxyacrylates include azoxystrobin, enestroburin (SYP-Z071) and picoxystrobin. The methoxycarbamates include pyraclostrobin. The oximinoacetates include kresoxim-methyl and trifloxystrobin. The oximinoacetamides include dimoxystrobin, metominostrobin, orysastrobin, α-[methoxyimino]-N-methyl-2-[[[1-[3-(trifluoromethyl)phenyl]ethoxy]imino]-methyl]benzeneacetamide and 2-[[[3-(2,6-dichlorophenyl)-1-methyl-2-propen-1-ylidene]-amino]oxy]methyl]-α-(methoxyimino)-N-methylbenzeneacetamide. The oxazolidinediones include famoxadone. The dihydrodioxazines include fluoxastrobin. The imidazolinones include fenamidone. The benzylcarbamates include pyribencarb.

(12) “Phenylpyrrole fungicides” (Fungicide Resistance Action Committee (FRAC) code 12) inhibit a MAP protein kinase associated with osmotic signal transduction in fungi. Fenpiclonil and fludioxonil are examples of this fungicide class.

(13) “Quinoline fungicides” (Fungicide Resistance Action Committee (FRAC) code 13) are proposed to inhibit signal transduction by affecting G-proteins in early cell signaling. They have been shown to interfere with germination and/or appressorium formation in fungi that cause powder mildew diseases. Quinoxyfen is an example of this class of fungicide.

(14) “Lipid peroxidation inhibitor fungicides” (Fungicide Resistance Action Committee (FRAC) code 14) are proposed to inhibit lipid peroxidation which affects membrane synthesis in fungi. Members of this class, such as etridiazole, may also affect other biological processes such as respiration and melanin biosynthesis. Lipid peroxidation fungicides include aromatic carbon and 1,2,4-thiadiazole fungicides. The aromatic carbon fungicides include biphenyl, chloroneb, dicloran, quintozene, tecnazene and tolclofos-methyl. The 1,2,4-thiadiazole fungicides include etridiazole.

(15) “Melanin biosynthesis inhibitors-reductase (MBI-R) fungicides” (Fungicide Resistance Action Committee (FRAC) code 16.1) inhibit the naphthal reduction step in melanin biosynthesis. Melanin is required for host plant infection by some fungi. Melanin biosynthesis inhibitors-reductase fungicides include isobenzofuranone, pyrroloquinolinone and triazolobenzothiazole fungicides. The isobenzofuranones include fthalide. The pyrroloquinolinones include pyroquilon. The triazolobenzothiazoles include tricyclazole.

(16) “Melanin biosynthesis inhibitors-dehydratase (MBI-D) fungicides” (Fungicide Resistance Action Committee (FRAC) code 16.2) inhibit scytalone dehydratase in melanin biosynthesis. Melanin in required for host plant infection by some fungi. Melanin biosynthesis inhibitors-dehydratase fungicides include cyclopropanecarboxamide, carboxamide and propionamide fungicides. The cyclopropanecarboxamides include carpropamid. The carboxamides include diclocymet. The propionamides include fenoxanil.

(17) “Hydroxyanilide fungicides (Fungicide Resistance Action Committee (FRAC) code 17) inhibit C4-demethylase which plays a role in sterol production. Examples include fenhexamid.

(18) “Squalene-epoxidase inhibitor fungicides” (Fungicide Resistance Action Committee (FRAC) code 18) inhibit squalene-epoxidase in ergosterol biosynthesis pathway. Sterols such as ergosterol are needed for membrane structure and function, making them essential for the development of functional cell walls. Therefore exposure to these fungicides results in abnormal growth and eventually death of sensitive fungi. Squalene-epoxidase inhibitor fungicides include thiocarbamate and allylamine fungicides. The thiocarbamates include pyributicarb. The allylamines include naftifine and terbinafine.

(19) “Polyoxin fungicides” (Fungicide Resistance Action Committee (FRAC) code 19) inhibit chitin synthase. Examples include polyoxin.

(20) “Phenylurea fungicides” (Fungicide Resistance Action Committee (FRAC) code

20) are proposed to affect cell division. Examples include pencycuron.

(21) “Quinone inside inhibitor (QiI) fungicides” (Fungicide Resistance Action Committee (FRAC) code 21) inhibit Complex III mitochondrial respiration in fungi by affecting ubiquinol reductase. Reduction of ubiquinol is blocked at the “quinone inside” (Qᵢ) site of the cytochrome bc₁ complex, which is located in the inner mitochondrial membrane of fungi. Inhibiting mitochondrial respiration prevents normal fungal growth and development. Quinone inside inhibitor fungicides include cyanoimidazole and sulfamoyltriazole fungicides. The cyanoimidazoles include cyazofamid. The sulfamoyltriazoles include amisulbrom.

(22) “Benzamide fungicides” (Fungicide Resistance Action Committee (FRAC) code 22) inhibit mitosis by binding to β-tubulin and disrupting microtubule assembly Inhibition of microtubule assembly can disrupt cell division, transport within the cell and cell structure. Examples include zoxamide.

(23) “Enopyranuronic acid antibiotic fungicides” (Fungicide Resistance Action Committee (FRAC) code 23) inhibit growth of fungi by affecting protein biosynthesis. Examples include blasticidin-S.

(24) “Hexopyranosyl antibiotic fungicides” (Fungicide Resistance Action Committee (FRAC) code 24) inhibit growth of fungi by affecting protein biosynthesis. Examples include kasugamycin.

(25) “Glucopyranosyl antibiotic: protein synthesis fungicides” (Fungicide Resistance Action Committee (FRAC) code 25) inhibit growth of fungi by affecting protein biosynthesis. Examples include streptomycin.

(26) “Glucopyranosyl antibiotic: trehalase and inositol biosynthesis fungicides” (Fungicide Resistance Action Committee (FRAC) code 26) inhibit trehalase in inositol biosynthesis pathway. Examples include validamycin.

(27) “Cyanoacetamideoxime fungicides (Fungicide Resistance Action Committee (FRAC) code 27) include cymoxanil.

(28) “Carbamate fungicides” (Fungicide Resistance Action Committee (FRAC) code 28) are considered multi-site inhibitors of fungal growth. They are proposed to interfere with the synthesis of fatty acids in cell membranes, which then disrupts cell membrane permeability. Propamacarb, propamacarb-hydrochloride, iodocarb, and prothiocarb are examples of this fungicide class.

(29) “Oxidative phosphorylation uncoupling fungicides” (Fungicide Resistance Action Committee (FRAC) code 29) inhibit fungal respiration by uncoupling oxidative phosphorylation. Inhibiting respiration prevents normal fungal growth and development. This class includes 2,6-dinitroanilines such as fluazinam, pyrimidonehydrazones such as ferimzone and dinitrophenyl crotonates such as dinocap, meptyldinocap and binapacryl.

(30) “Organo tin fungicides” (Fungicide Resistance Action Committee (FRAC) code 30) inhibit adenosine triphosphate (ATP) synthase in oxidative phosphorylation pathway. Examples include fentin acetate, fentin chloride and fentin hydroxide.

(31) “Carboxylic acid fungicides” (Fungicide Resistance Action Committee (FRAC) code 31) inhibit growth of fungi by affecting deoxyribonucleic acid (DNA) topoisomerase type II (gyrase). Examples include oxolinic acid.

(32) “Heteroaromatic fungicides” (Fungicide Resistance Action Committee (FRAC) code 32) are proposed to affect DNA/ribonucleic acid (RNA) synthesis. Heteroaromatic fungicides include isoxazole and isothiazolone fungicides. The isoxazoles include hymexazole and the isothiazolones include octhilinone.

(33) “Phosphonate fungicides” (Fungicide Resistance Action Committee (FRAC) code 33) include phosphorus acid and its various salts, including fosetyl-aluminum.

(34) “Phthalamic acid fungicides” (Fungicide Resistance Action Committee (FRAC) code 34) include teclofthalam.

(35) “Benzotriazine fungicides” (Fungicide Resistance Action Committee (FRAC) code 35) include triazoxide.

(36) “Benzene-sulfonamide fungicides” (Fungicide Resistance Action Committee (FRAC) code 36) include flusulfamide.

(37) “Pyridazinone fungicides” (Fungicide Resistance Action Committee (FRAC) code 37) include diclomezine.

(38) “Thiophene-carboxamide fungicides” (Fungicide Resistance Action Committee (FRAC) code 38) are proposed to affect ATP production. Examples include silthiofam.

(39) “Pyrimidinamide fungicides” (Fungicide Resistance Action Committee (FRAC) code 39) inhibit growth of fungi by affecting phospholipid biosynthesis and include diflumetorim.

(40) “Carboxylic acid amide (CAA) fungicides” (Fungicide Resistance Action Committee (FRAC) code 40) are proposed to inhibit phospholipid biosynthesis and cell wall deposition. Inhibition of these processes prevents growth and leads to death of the target fungus. Carboxylic acid amide fungicides include cinnamic acid amide, valinamide carbamate and mandelic acid amide fungicides. The cinnamic acid amides include dimethomorph and flumorph. The valinamide carbamates include benthiavalicarb, benthiavalicarb-isopropyl, iprovalicarb and valiphenal. The mandelic acid amides include mandipropamid, N-[2-[4-[[3-(4-chlorophenyl)-2-propyn-1-yl]oxy]-3-methoxyphenyl]ethyl]-3-methyl-2-[(methylsulfonyl)amino]butanamide and N-[2-[4-[[3-(4-chlorophenyl)-2-propyn-1-yl]oxy]-3-methoxyphenyl]ethyl]-3-methyl-2-[(ethylsulfonyl)amino]butanamide.

(41) “Tetracycline antibiotic fungicides” (Fungicide Resistance Action Committee (FRAC) code 41) inhibit growth of fungi by affecting complex 1 nicotinamide adenine dinucleotide (NADH) oxidoreductase. Examples include oxytetracycline.

(42) “Thiocarbamate fungicides (b42)” (Fungicide Resistance Action Committee (FRAC) code 42) include methasulfocarb.

(43) “Benzamide fungicides” (Fungicide Resistance Action Committee (FRAC) code 43) inhibit growth of fungi by delocalization of spectrin-like proteins. Examples include acylpicolide fungicides such as fluopicolide and fluopyram.

(44) “Host plant defense induction fungicides” (Fungicide Resistance Action Committee (FRAC) code P) induce host plant defense mechanisms. Host plant defense induction fungicides include benzo-thiadiazole, benzisothiazole and thiadiazole-carboxamide fungicides. The benzo-thiadiazoles include acibenzolar-5-methyl. The benzisothiazoles include probenazole. The thiadiazole-carboxamides include tiadinil and isotianil.

(45) “Multi-site contact fungicides” inhibit fungal growth through multiple sites of action and have contact/preventive activity. This class of fungicides includes: (45.1) “copper fungicides” (Fungicide Resistance Action Committee (FRAC) code M1)”, (45.2) “sulfur fungicides” (Fungicide Resistance Action Committee (FRAC) code M2), (45.3) “dithiocarbamate fungicides” (Fungicide Resistance Action Committee (FRAC) code M3), (45.4) “phthalimide fungicides” (Fungicide Resistance Action Committee (FRAC) code M4), (45.5) “chloronitrile fungicides” (Fungicide Resistance Action Committee (FRAC) code M5), (45.6) “sulfamide fungicides” (Fungicide Resistance Action Committee (FRAC) code M6), (45.7) “guanidine fungicides” (Fungicide Resistance Action Committee (FRAC) code M7), (45.8) “triazine fungicides” (Fungicide Resistance Action Committee (FRAC) code M8) and (45.9) “quinone fungicides” (Fungicide Resistance Action Committee (FRAC) code M9). “Copper fungicides” are inorganic compounds containing copper, typically in the copper(II) oxidation state; examples include copper oxychloride, copper sulfate and copper hydroxide, including compositions such as Bordeaux mixture (tribasic copper sulfate). “Sulfur fungicides” are inorganic chemicals containing rings or chains of sulfur atoms; examples include elemental sulfur. “Dithiocarbamate fungicides” contain a dithiocarbamate molecular moiety; examples include mancozeb, metiram, propineb, ferbam, maneb, thiram, zineb and ziram. “Phthalimide fungicides” contain a phthalimide molecular moiety; examples include folpet, captan and captafol. “Chloronitrile fungicides” contain an aromatic ring substituted with chloro and cyano; examples include chlorothalonil. “Sulfamide fungicides” include dichlofluanid and tolyfluanid. “Guanidine fungicides” include dodine, guazatine, iminoctadine albesilate and iminoctadine triacetate. “Triazine fungicides” include anilazine. “Quinone fungicides” include dithianon.

(46) “Fungicides other than fungicides of classes (1) through (45)” include certain fungicides whose mode of action may be unknown. These include: (46.1) “thiazole carboxamide fungicides” (Fungicide Resistance Action Committee (FRAC) code U5), (46.2) “phenyl-acetamide fungicides” (Fungicide Resistance Action Committee (FRAC) code U6), (46.3) “quinazolinone fungicides” (Fungicide Resistance Action Committee (FRAC) code U7) and (46.4) “benzophenone fungicides” (Fungicide Resistance Action Committee (FRAC) code U8). The thiazole carboxamides include ethaboxam. The phenyl-acetamides include cyflufenamid and N-[[(cyclopropylmethoxy)amino][6-(difluoromethoxy)-2,3-difluorophenyl]-methyleneThenzeneacetamide. The quinazolinones include proquinazid and 2-butoxy-6-iodo-3-propyl-4H-1-benzopyran-4-one. The benzophenones include metrafenone. The (b46) class also includes bethoxazin, neo-asozin (ferric methanearsonate), pyrrolnitrin, quinomethionate, N-[2-[4-[[3-(4-chlorophenyl)-2-propyn-1-yl]oxy]-3-methoxyphenyl]ethyl]-3-methyl-2-[(methylsulfonyl)amino]butanamide, N-[2-[4-[[3-(4-chlorophenyl)-2-propyn-1-yl]oxy]-3-methoxyphenyl]ethyl]-3-methyl-2-[(ethylsulfonyl)amino]-butanamide, 2-[[2-fluoro-5-(trifluoromethyl)phenyl]thio]-2-[3-(2-methoxyphenyl)-2-thiazo-lidinylidene]acetonitrile, 3-[5-(4-chlorophenyl)-2,3-dimethyl-3-isoxazolidinyl]pyridine, 4-fluorophenyl N-[1-[[[1-(4-cyanophenyl)ethyl]sulfonyl]methyl]propyl]carbamate, 5-chloro-6-(2,4,6-trifluorophenyl)-7-(4-methylpiperidin-1-yl) [1,2,4]triazolo[1,5-a]pyrimidine, N-(4-chloro-2-nitrophenyl)-N-ethyl-4-methylbenzenesulfonamide, N-[[(cyclopropylmethoxy)-amino][6-(difluoromethoxy)-2,3-difluorophenyl]methylene]benzeneacetamide, N′-[4-[4-chloro-3-(trifluoromethyl)phenoxy]-2,5-dimethylphenyl]-N-ethyl-N-methylmethanimidamide and 1-[(2-propenylthio) carbonyl]-2-(1-methylethyl)-4-(2-methylphenyl)-5-amino-1H-pyrazol-3-one.

Therefore of note is a mixture (i.e. composition) comprising a compound of Formula 1 and at least one fungicidal compound selected from the group consisting of the aforedescribed classes (1) through (46). Also of note is a composition comprising said mixture (in fungicidally effective amount) and further comprising at least one additional component selected from the group consisting of surfactants, solid diluents and liquid diluents. Of particular note is a mixture (i.e. composition) comprising a compound of Formula 1 and at least one fungicidal compound selected from the group of specific compounds listed above in connection with classes (1) through (46). Also of particular note is a composition comprising said mixture (in fungicidally effective amount) and further comprising at least one additional surfactant selected from the group consisting of surfactants, solid diluents and liquid diluents.

Examples of other biologically active compounds or agents with which compounds of this invention can be formulated are: insecticides such as abamectin, acephate, acetamiprid, amidoflumet (S-1955), avermectin, azadirachtin, azinphos-methyl, bifenthrin, bifenazate, 3-bromo-1-(3-chloro-2-pyridinyl)-N-[4-cyano-2-methyl-6-[(methylamino)carbonyl]phenyl]-1H-pyrazole-5-carboxamide, buprofezin, carbofuran, cartap, chlorantraniliprole (DPX-E2Y45), chlorfenapyr, chlorfluazuron, chlorpyrifos, chlorpyrifos-methyl, chromafenozide, clothianidin, cyflumetofen, cyfluthrin, beta-cyfluthrin, cyhalothrin, lambda-cyhalothrin, cypermethrin, cyromazine, deltamethrin, diafenthiuron, diazinon, dieldrin, diflubenzuron, dimefluthrin, dimethoate, dinotefuran, diofenolan, emamectin, endosulfan, esfenvalerate, ethiprole, fenothiocarb, fenoxycarb, fenpropathrin, fenvalerate, fipronil, flonicamid, flubendiamide, flucythrinate, tau-fluvalinate, flufenerim (UR-50701), flufenoxuron, fonophos, halofenozide, hexaflumuron, hydramethylnon, imidacloprid, indoxacarb, isofenphos, lufenuron, malathion, metaflumizone, metaldehyde, methamidophos, methidathion, methomyl, methoprene, methoxychlor, metofluthrin, monocrotophos, methoxyfenozide, nitenpyram, nithiazine, novaluron, noviflumuron (XDE-007), oxamyl, parathion, parathion-methyl, permethrin, phorate, phosalone, phosmet, phosphamidon, pirimicarb, profenofos, profluthrin, pymetrozine, pyrafluprole, pyrethrin, pyridalyl, pyrifluquinazon, pyriprole, pyriproxyfen, rotenone, ryanodine, spinetoram, spinosad, spirodiclofen, spiromesifen (BSN 2060), spirotetramat, sulprofos, tebufenozide, teflubenzuron, tefluthrin, terbufos, tetrachlorvinphos, thiacloprid, thiamethoxam, thiodicarb, thiosultap-sodium, tralomethrin, triazamate, trichlorfon and triflumuron; and biological agents including entomopathogenic bacteria, such as Bacillus thuringiensis subsp. aizawai, Bacillus thuringiensis subsp. kurstaki, and the encapsulated delta-endotoxins of Bacillus thuringiensis (e.g., Cellcap, MPV, MPVII); entomopathogenic fungi, such as green muscardine fungus; and entomopathogenic virus including baculovirus, nucleopolyhedro virus (NPV) such as HzNPV, AfNPV; and granulosis virus (GV) such as CpGV.

Compounds of this invention and compositions thereof can be applied to plants genetically transformed to express proteins toxic to invertebrate pests (such as Bacillus thuringiensis delta-endotoxins). The effect of the exogenously applied fungicidal compounds of this invention may be synergistic with the expressed toxin proteins.

General references for agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U.K., 2003 and The BioPesticide Manual, 2nd Edition, L. G. Copping, Ed., British Crop Protection Council, Farnham, Surrey, U.K., 2001.

For embodiments where one or more of these various mixing partners are used, the weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of diseases controlled beyond the spectrum controlled by the compound of Formula 1 alone.

In certain instances, combinations of a compound of this invention with other biologically active (particularly fungicidal) compounds or agents (i.e. active ingredients) can result in a greater-than-additive (i.e. synergistic) effect. Reducing the quantity of active ingredients released in the environment while ensuring effective pest control is always desirable. When synergism of fungicidal active ingredients occurs at application rates giving agronomically satisfactory levels of fungal control, such combinations can be advantageous for reducing crop production cost and decreasing environmental load.

Of note is a combination of a compound of Formula 1 with at least one other fungicidal active ingredient. Of particular note is such a combination where the other fungicidal active ingredient has different site of action from the compound of Formula 1. In certain instances, a combination with at least one other fungicidal active ingredient having a similar spectrum of control but a different site of action will be particularly advantageous for resistance management. Thus, a composition of the present invention can further comprise a biologically effective amount of at least one additional fungicidal active ingredient having a similar spectrum of control but a different site of action.

Of particular note are compositions which in addition to compound of Formula 1 include at least one compound selected from the group consisting of (1) alkylenebis(dithiocarbamate) fungicides; (2) cymoxanil; (3) phenylamide fungicides; (4) pyrimidinone fungicides; (5) chlorothalonil; (6) carboxamides acting at complex II of the fungal mitochondrial respiratory electron transfer site; (7) quinoxyfen; (8) metrafenone; (9) cyflufenamid; (10) cyprodinil; (11) copper compounds; (12) phthalimide fungicides; (13) fosetyl-aluminum; (14) benzimidazole fungicides; (15) cyazofamid; (16) fluazinam; (17) iprovalicarb; (18) propamocarb; (19) validomycin; (20) dichlorophenyl dicarboximide fungicides; (21) zoxamide; (22) fluopicolide; (23) mandipropamid; (24) carboxylic acid amides acting on phospholipid biosynthesis and cell wall deposition; (25) dimethomorph; (26) non-DMI sterol biosynthesis inhibitors; (27) inhibitors of demethylase in sterol biosynthesis; (28) bc₁ complex fungicides; and salts of compounds of (1) through (28).

Further descriptions of classes of fungicidal compounds are provided below.

Pyrimidinone fungicides (group (4)) include compounds of Formula A1

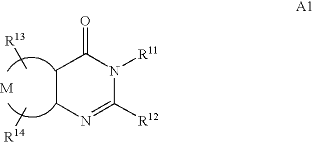

wherein M forms a fused phenyl, thiophene or pyridine ring; R¹¹ is C₁-C₆ alkyl; R¹² is C₁-C₆ alkyl or C₁-C₆ alkoxy; R¹³ is halogen; and R¹⁴ is hydrogen or halogen.

Pyrimidinone fungicides are described in PCT Patent Application Publication WO 94/26722 and U.S. Pat. Nos. 6,066,638, 6,245,770, 6,262,058 and 6,277,858. Of note are pyrimidinone fungicides selected from the group: 6-bromo-3-propyl-2-propyloxy-4(3H)-quinazolinone, 6,8-diiodo-3-propyl-2-propyloxy-4(3H)-quinazolinone, 6-iodo-3-propyl-2-propyloxy-4(3H)-quinazolinone (proquinazid), 6-chloro-2-propoxy-3-propylthieno[2,3-d]pyrimidin-4(3H)-one, 6-bromo-2-propoxy-3-propylthieno[2,3-d]pyrimidin-4(3H)-one, 7-bromo-2-propoxy-3-propylthieno[3,2-d]pyrimidin-4(3H)-one, 6-bromo-2-propoxy-3-propylpyrido[2,3-d]pyrimidin-4(3H)-one, 6,7-dibromo-2-propoxy-3-propylthieno[3,2-d]pyrimidin-4(3H)-one, and 3-(cyclopropylmethyl)-6-iodo-2-(propylthio)pyrido-[2,3-d]pyrimidin-4(3H)-one.

Sterol biosynthesis inhibitors (group (27)) control fungi by inhibiting enzymes in the sterol biosynthesis pathway. Demethylase-inhibiting fungicides have a common site of action within the fungal sterol biosynthesis pathway, involving inhibition of demethylation at position 14 of lanosterol or 24-methylene dihydrolanosterol, which are precursors to sterols in fungi. Compounds acting at this site are often referred to as demethylase inhibitors, DMI fungicides, or DMIs. The demethylase enzyme is sometimes referred to by other names in the biochemical literature, including cytochrome P-450 (14DM). The demethylase enzyme is described in, for example, J. Biol. Chem. 1992, 267, 13175-79 and references cited therein. DMI fungicides are divided between several chemical classes: azoles (including triazoles and imidazoles), pyrimidines, piperazines and pyridines. The triazoles include azaconazole, bromuconazole, cyproconazole, difenoconazole, diniconazole (including diniconazole-M), epoxiconazole, etaconazole, fenbuconazole, fluquinconazole, flusilazole, flutriafol, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole and uniconazole. The imidazoles include clotrimazole, econazole, imazalil, isoconazole, miconazole, oxpoconazole, prochloraz and triflumizole. The pyrimidines include fenarimol, nuarimol and triarimol. The piperazines include triforine. The pyridines include buthiobate and pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

bc₁ Complex Fungicides (group 28) have a fungicidal mode of action which inhibits the bc₁ complex in the mitochondrial respiration chain. The bc₁ complex is sometimes referred to by other names in the biochemical literature, including complex III of the electron transfer chain, and ubihydroquinone:cytochrome c oxidoreductase. This complex is uniquely identified by Enzyme Commission number EC1.10.2.2. The bc₁ complex is described in, for example, J. Biol. Chem. 1989, 264, 14543-48; Methods Enzymol. 1986, 126, 253-71; and references cited therein. Strobilurin fungicides such as azoxystrobin, dimoxystrobin, enestroburin (SYP-Z071), fluoxastrobin, kresoxim-methyl, metominostrobin, orysastrobin, picoxystrobin, pyraclostrobin and trifloxystrobin are known to have this mode of action (H. Sauter et al., Angew. Chem. Int. Ed. 1999, 38, 1328-1349). Other fungicidal compounds that inhibit the bc₁ complex in the mitochondrial respiration chain include famoxadone and fenamidone.

Alkylenebis(dithiocarbamate)s (group (1)) include compounds such as mancozeb, maneb, propineb and zineb. Phenylamides (group (3)) include compounds such as metalaxyl, benalaxyl, furalaxyl and oxadixyl. Carboxamides (group (6)) include compounds such as boscalid, carboxin, fenfuram, flutolanil, furametpyr, mepronil, oxycarboxin, thifluzamide, penthiopyrad and N-[2-(1,3-dimethylbutyl)phenyl]-5-fluoro-1,3-dimethyl-1H-pyrazole-4-carboxamide (PCT Patent Publication WO 2003/010149), and are known to inhibit mitochondrial function by disrupting complex II (succinate dehydrogenase) in the respiratory electron transport chain. Copper compounds (group (11)) include compounds such as copper oxychloride, copper sulfate and copper hydroxide, including compositions such as Bordeaux mixture (tribasic copper sulfate). Phthalimides (group (12)) include compounds such as folpet and captan. Benzimidazole fungicides (group (14)) include benomyl and carbendazim. Dichlorophenyl dicarboximide fungicides (group (20)) include chlozolinate, dichlozoline, iprodione, isovaledione, myclozolin, procymidone and vinclozolin.

Non-DMI sterol biosynthesis inhibitors (group (26)) include morpholine and piperidine fungicides. The morpholines and piperidines are sterol biosynthesis inhibitors that have been shown to inhibit steps in the sterol biosynthesis pathway at a point later than the inhibitions achieved by the DMI sterol biosynthesis (group (27)). The morpholines include aldimorph, dodemorph, fenpropimorph, tridemorph and trimorphamide. The piperidines include fenpropidin

Of further note are combinations of compounds of Formula 1 with azoxystrobin, kresoxim-methyl, trifloxystrobin, pyraclostrobin, picoxystrobin, dimoxystrobin, metominostrobin/fenominostrobin, carbendazim, chlorothalonil, quinoxyfen, metrafenone, cyflufenamid, fenpropidine, fenpropimorph, bromuconazole, cyproconazole, difenoconazole, epoxiconazole, fenbuconazole, flusilazole, hexaconazole, ipconazole, metconazole, penconazole, propiconazole, proquinazid, prothioconazole, tebuconazole, triticonazole, famoxadone, prochloraz, penthiopyrad and boscalid (nicobifen).

Preferred for better control of plant diseases caused by fungal plant pathogens (e.g., lower use rate or broader spectrum of plant pathogens controlled) or resistance management are mixtures of a compound of this invention with a fungicide selected from the group: azoxystrobin, kresoxim-methyl, trifloxystrobin, pyraclostrobin, picoxystrobin, dimoxystrobin, metominostrobin/fenominostrobin, quinoxyfen, metrafenone, cyflufenamid, fenpropidine, fenpropimorph, cyproconazole, epoxiconazole, flusilazole, metconazole, propiconazole, proquinazid, prothioconazole, tebuconazole, triticonazole, famoxadone and penthiopyrad.

Specifically preferred mixtures (compound numbers refer to compounds in Index Table A) are selected from the group: combinations of Compound 1, Compound 2, Compound 3, Compound 4 or Compound 5 with azoxystrobin, combinations of Compound 1, Compound 2, Compound 3, Compound 4 or Compound 5 with trifloxystrobin, combinations of Compound 1, Compound 2, Compound 3, Compound 4 or Compound 5 with pyraclostrobin, combinations of Compound 1, Compound 2, Compound 3, Compound 4 or Compound 5 with picoxystrobin, combinations of Compound 1, Compound 2, Compound 3, Compound 4 or Compound 5 with dimoxystrobin, combinations of Compound 1, Compound 2, Compound 3, Compound 4 or Compound 5 with metominostrobin/fenominostrobin, combinations of Compound 1, Compound 2, Compound 3, Compound 4 or Compound 5 with quinoxyfen, combinations of Compound 1, Compound 2, Compound 3, Compound 4 or Compound 5 with metrafenone, combinations of Compound 1, Compound 2, Compound 3, Compound 4 or Compound 5 with cyflufenamid, combinations of Compound 1, Compound 2, Compound 3, Compound 4 or Compound 5 with fenpropidine, combinations of Compound 1, Compound 2, Compound 3, Compound 4 or Compound 5 with fenpropimorph, combinations of C Compound 1, Compound 2, Compound 3, Compound 4 or Compound 5 with cyproconazole, combinations of Compound 1, Compound 2, Compound 3, Compound 4 or Compound 5 with epoxiconazole, combinations of Compound 1, Compound 2, Compound 3, Compound 4 or Compound 5 with flusilazole, combinations of Compound 1, Compound 2, Compound 3, Compound 4 or Compound 5 with metconazole, combinations of Compound 1, Compound 2, Compound 3, Compound 4 or Compound 5 with propiconazole, combinations of Compound 1, Compound 2, Compound 3, Compound 4 or Compound 5 with prothioconazole, combinations of Compound 1, Compound 2, Compound 3, Compound 4 or Compound 5 with tebuconazole, combinations of Compound 1, Compound 2, Compound 3, Compound 4 or Compound 5 with triticonazole, combinations of Compound 1, Compound 2, Compound 3, Compound 4 or Compound 5 with famoxadone, and combinations of C Compound 1, Compound 2, Compound 3, Compound 4 or Compound 5 with penthiopyrad.

The rate of application required for effective control (i.e. “biologically effective amount”) will depend on such factors as the plant diseases to be controlled, the location, time of year, host crop, ambient moisture, temperature, and the like. One skilled in the art can easily determine through simple experimentation the biologically effective amount necessary for the desired level of plant disease control.

The following Tests demonstrate the control efficacy of compounds of this invention on specific pathogens. The disease control afforded by the mixtures is not limited, however, to the pathogenic fungi species exemplified. See Index Table A for compound descriptions of Formula 1. In Index Table A the abbreviation Me means methyl. The abbreviation “Ex.” stands for “Example” and is followed by a number indicating in which example the compound is prepared. In the following table a dash (“-”) in the (R¹)ₙ indicates n is 0 and hydrogen is present at all available positions.

INDEX TABLE A

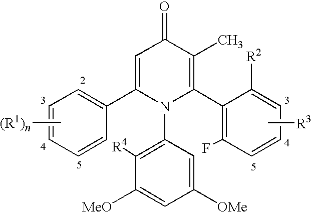

| Compound | (R¹)ₙ | R² | R³ | R⁴ | ¹H NMR |
|---|---|---|---|---|---|
| 1 (Ex. 1) | 4-F | F | 4-F | Cl | ** |
| 2 | — | F | 4-F | Cl | * |
| 3 | — | F | 4-F | H | * |
| 4 | 2-F | F | 4-F | H | * |
| 5 | 4-F | F | 4-F | H | * |

* See Index Table B for ¹H NMR data.
** See synthesis example for ¹H NMR data.

INDEX TABLE B

| Compd. No. | ¹H NMR Data (CDCl₃ solution unless indicated otherwise)^{a} |
|---|---|
| 2 | δ 7.25-7.16 (m, 6H), 6.63-6.55 (m, 2H), 6.39 (t, J = 3 Hz, 1H), 6.20 (d, J = 2.4 Hz, 1H), |
| | 3.68 (s, 3H), 3.63 (s, 3H), 1.91 (s, 3H). |
| 3 | δ 7.22-7.17 (m, 5H), 6.60-6.54 (m, 3H), 6.11 (s, 3H), 3.55 (s, 6H), 1.90 (s, 3H). |
| 4 | δ 7.45-7.40 (m, 1H), 7.35-7.3 (m, 1H), 7.27-7.23 (m, 1H), 7.19-7.15 (m, 1H), 6.72 (s, 1H), |
| | 6.58-6.54 (t, J = 8 Hz, 2H), 6.38 (s, 2H), 6.34-6.33 (m, 1H), 3.71 (s, 6H), 1.67 (s, 3H). |
| 5 | δ 7.18-7.14 (m, 2H), 6.91-6.87 (m, 2H), 6.62-6.56 (m, 2H), 6.49 (s, 1H), 6.14-6.10 (m, 3H), |
| | 3.50 (s, 6H) 1.89 (s, 3H). |

^{a1}H NMR data are in ppm downfield from tetramethylsilane. Couplings are designated by (s)-singlet, (d)-doublet, (t)-triplet, (m)-multiplet.

### Biological Examples of the Invention

General protocol for preparing test suspensions for Tests A-F: the test compounds were first dissolved in acetone in an amount equal to 3% of the final volume and then suspended at the desired concentration (in ppm) in acetone and purified water (50/50 mix by volume) containing 250 ppm of the surfactant Trem® 014 (polyhydric alcohol esters). The resulting test suspensions were then used in Tests A-F. Spraying a 200 ppm test suspension to the point of run-off on the test plants was the equivalent of a rate of 500 g/ha.

### Test A

The test suspension was sprayed to the point of run-off on wheat seedlings. The following day the seedlings were inoculated with a spore dust of Erysiphe graminis f. sp. tritici (the causal agent of wheat powdery mildew) and incubated in a growth chamber at 20° C. for 8 days, after which time disease ratings were made.

### Test B

The test suspension was sprayed to the point of run-off on wheat seedlings. The following day the seedlings were inoculated with a spore suspension of Puccinia recondite f. sp. tritici (the causal agent of wheat leaf rust) and incubated in a saturated atmosphere at 20° C. for 24 h, and then moved to a growth chamber at 20° C. for 7 days, after which time disease ratings were made.

### Test C

The test suspension was sprayed to the point of run-off on wheat seedlings. The following day the seedlings were inoculated with a spore suspension of Septoria tritici (the causal agent of wheat leaf blotch) and incubated in a saturated atmosphere at 20° C. for 48 h, and then moved to a growth chamber at 20° C. for 19 days, after which time disease ratings were made.

### Test D

The test suspension was sprayed to the point of run-off on wheat seedlings. The following day the seedlings were inoculated with a spore suspension of Septoria nodorum (the causal agent of wheat glume blotch) and incubated in a saturated atmosphere at 20° C. for 48 h, and then moved to a growth chamber at 20° C. for 7 days, after which time disease ratings were made.

### Test E

The test suspension was sprayed to the point of run-off on tomato seedlings. The following day the seedlings were inoculated with a spore suspension of Alternaria solani (the causal agent of tomato early blight) and incubated in a saturated atmosphere at 27° C. for 48 h, and then moved to a growth chamber at 20° C. for 5 days, after which time disease ratings were made.

### Test F

The test suspension was sprayed to the point of run-off on tomato seedlings. The following day the seedlings were inoculated with a spore suspension of Botrytis cinerea (the causal agent of tomato Botrytis) and incubated in a saturated atmosphere at 20° C. for 48 h, and then moved to a growth chamber at 24° C. for 3 days, after which time disease ratings were made.

Results for Tests A-F are given in Table A. In the Table, a rating of 100 indicates 100% disease control and a rating of 0 indicates no disease control (relative to the controls).

TABLE A

| Cmpd No. | Test A | Test B | Test C | Test D | Test E | Test F |
|---|---|---|---|---|---|---|
| 1 | 99 | 100 | 97 | 100 | 100 | 98 |
| 2 | 99 | 100 | 98 | 100 | 99 | 99 |
| 3 | 99 | 100 | 97 | 100 | 100 | 100 |
| 4 | 100 | 100 | 98 | 100 | 100 | 99 |
| 5 | 100 | 100 | 97 | 100 | 100 | 99 |

What is claimed is:

1. A compound selected from Formula 1, N-oxides, and salts thereof, 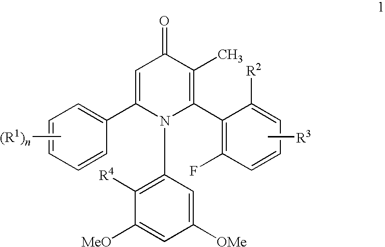
wherein
each R¹ is independently halogen, cyano, hydroxy, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₃-C₆ cycloalkenyl, C₂-C₆ alkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₃-C₆ dialkylaminoalkyl, C₂-C₆ alkylcarbonyl, C₂-C₆ haloalkylcarbonyl, C₂-C₆ alkoxycarbonyl, C₂-C₆ alkylaminocarbonyl, C₃-C₈ dialkylaminocarbonyl, C₂-C₆ cyanoalkyl, C₁-C₆ hydroxyalkyl, C₁-C₆ alkoxy, C₁-C₆ haloalkoxy, C₃-C₆ cycloalkoxy, C₃-C₆ halocycloalkoxy, C₂-C₆ alkoxyalkoxy, C₃-C₆ alkoxycarbonylalkyl, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₁-C₆ alkylamino, C₂-C₆ dialkylamino, C₂-C₆ haloalkylamino, C₂-C₆ halodialkylamino, C₂-C₆ alkylcarbonylamino, C₂-C₆ haloalkylcarbonylamino, C₁-C₆ alkylsulfonylamino or C₁-C₆ haloalkylsulfonylamino;
R² is F or Cl;
R³ is halogen or methoxy;
R⁴ is H or halogen; and
n is an integer selected from 0, 1, 2, 3, 4 and 5.

2. A compound of claim 1 wherein:
each R¹ is independently halogen, cyano, nitro, C₁-C₆ alkyl, C₂-C₆ alkenyl, C₂-C₆ alkynyl, C₁-C₆ haloalkyl, C₂-C₆ alkoxyalkyl, C₂-C₆ alkylcarbonyl, C₂-C₆ alkoxycarbonyl, C₂-C₆ alkylaminocarbonyl, C₃-C₈ dialkylaminocarbonyl, C₂-C₆ cyanoalkyl, C₁-C₆ hydroxyalkyl, C₁-C₆ alkoxy, C₁-C₆ alkylthio, C₁-C₆ alkylsulfinyl, C₁-C₆ alkylsulfonyl, C₁-C₆ alkylamino, C₂-C₆ dialkylamino or C₂-C₆ alkylcarbonylamino.

3. A compound of claim 2 wherein:
each R¹ is independently is halogen, C₁-C₆ alkyl, C₂-C₆ alkenyl, C₁-C₆ haloalkyl, C₂-C₆ alkylcarbonyl, C₂-C₆ alkoxycarbonyl or C₁-C₆ alkoxy;
R³ is Cl, F, Br or methoxy; and
R⁴ is H, Cl, F or Br.

4. A compound of claim 3 wherein:
each R¹ is independently halogen, C₁-C₆ alkyl or C₁-C₆ alkoxy;
R³ is F or methoxy; and
R⁴ is Cl or F.

5. A compound of claim 4 wherein:
R² is F.
R³ is F or methoxy;
R⁴ is Cl or F; and
n is 0 or 1.

6. A compound of claim 5 wherein:
R³ is attached at the para position.

7. A compound of claim 6 wherein:
R³ is F.

8. A compound of claim 6 wherein:
R³ is methoxy.

9. A fungicidal composition comprising (a) a compound of claim 1; and (b) at least one other fungicide.

10. A fungicidal composition comprising (a) a compound of claim 1; and (b) at least one additional component selected from the group consisting of surfactants, solid diluents and liquid diluents.

11. A method for controlling plant diseases caused by fungal plant pathogens comprising applying to the plant or portion thereof, or to the plant seed, a fungicidally effective amount of a compound of claim 1.
